(19) United States
(12) Patent Application Publication
(10) Pub. No.: US 2019/0145916 A1
(43) Pub. Date: May 16, 2019

(54) Compact, Low Cost Apparatus for Testing of Production and Counterfeit Pharmaceuticals and Other Crystalline Materials

(71) Applicant: XRD by Design LLC, Canonsburg, PA (US)

(72) Inventor: William E. Mayo, Canonsburg, PA (US)

(73) Assignee: XRD by Design LLC, Canonsburg, PA (US)

(21) Appl. No.: 16/010,623

(22) Filed: Jun. 18, 2018

Related U.S. Application Data
(60) Provisional application No. 62/587,005, filed on Nov. 16, 2017.

(51) Int. Cl. G01N 23/207; G01N 23/20091; G01N 33/15

(52) U.S. Cl. CPC G01N 23/2076; B33Y 80/00; G01N 33/15; G01N 23/20091

(57) ABSTRACT

A compact, low-cost system for the detection of counterfeit or sub-potency pharmaceuticals is implemented by use of a low power X-ray source, an incident collimator containing a series of concentric, non-parallel slits, receiving collimators containing a series of concentric, non-parallel slits, additional collimators to limit tangential divergence and a single, near room temperature energy dispersive detector that sums the plurality of diffracted x-ray beams. In this system, the tradeoff between spectral resolving power and the diffracted intensity is eliminated.

Also provided are methods to determine the optimal diffraction angle for a given test material, determine the instrument geometry and design parameters, and assess the system performance and sensitivity to alignment errors.

### CROSS-REFERENCE TO RELATED APPLICATIONS

This application claims the benefit of U.S. Provisional Application No. 62/587,005, filed on Nov. 16, 2017, the contents of which are incorporated by reference herein, in their entireties and for all purposes.

### BACKGROUND

### Field of the Invention

The invention pertains to the field of X-ray diffraction and particularly to the design and use of collimator designs to enhance the diffracted intensity without degradation of the system's spectral resolving power. These performance improvements permit the development of a compact, low cost apparatus for the detection of counterfeit pharmaceuticals and quality control testing of production materials such as pharmaceuticals and other materials.

### Background

Energy Dispersive X-ray Diffraction (EDXRD) has been in use since 1968 but is very limited in its scope and usefulness compared to the more common Angular Dispersive X-ray Diffraction (ADXRD) method. Both methods rely on the well-known phenomenon of X-ray diffraction governed by Bragg's Law

nλ=2d sin(Θ)

where n=an integer indicating the reflection order, λ=the x-ray wavelength, d=spacing between parallel planes in a crystal and Θ=one half of the angle between the incident and diffracted beams. The goal of any x-ray diffraction (XRD) method is to determine the set of d values representing the various interplanar crystalline spacings of a test sample using suitable values of λ and Θ. ADXRD for example, uses a constant λ value to produce a diffraction pattern of individual peaks as a function of the variable angle Θ. Each peak thus generated corresponds to a specific d value, and the total set of d values comprises a unique fingerprint of the test sample. By contrast, EDXRD uses a fixed angle Θ to generate a diffraction pattern of individual peaks as a function of the variable wavelength λ (or its related photon energy E), thus producing an equivalent set of d values as the ADXRD method. Although the set of d values obtained by the ADXRD and EDXRD methods will be the same to within the experimental error, the obtained diffraction patterns will be visually quite different, especially with regard to the intensities and peak breadths.

ADXRD utilizes a semi-monochromatic X-ray beam with a fixed wavelength typically between 0.07 nm and 0.22 nm, a detector that is non-energy discriminating, and collimating optics consisting of narrow slits or plates that limit the size and divergence of both the incident and diffracted beams. The resulting diffraction patterns typically contain numerous sharp peaks with good intensities that permit rapid data collection and analysis. One feature of ADXRD is that the information obtained from the diffraction pattern is limited to the near surface region of the test samples, owing to the low penetrating power of the low energy incident X-ray beam. Typically, the penetration depth of the incident beam is of the order of 1-100 μm for the most common radiation (Cu tube with λ=0.154 nm or E≈8 keV). By contrast, EDXRD utilizes an incident X-ray beam with much higher energies, which results in greater penetrating power of up to 1,000,000 μm, depending on the chemical composition and density of the test object. Due to this great difference in penetrating power, ADXRD is typically used in a reflection mode to analyze the near-surface region while EDXRD is most often used in a transmission mode to analyze the interior volume.

One of the consequences of the great penetrating power of the EDXRD incident X-ray beam is that the diffracted signal arriving at the detector comes from numerous points within the sample along the entire incident beam path. This factor causes a severe broadening of the diffraction peaks and a concomitant loss of spectral resolving power, which then degrades the ability to extract the information necessary for material analysis. It is possible to partially compensate for this effect by using very narrow collimator slits that limit the angular divergence of the incident and diffracted beams, but this significantly reduces the diffracted beam intensity. Thus, for a given system input power, the resolving power of a conventional EDXRD system and the diffracted beam intensity are inversely related.

In an EDXRD system, when the intensity is low, the signal-to-noise (S/N) ratio is low, making it difficult to distinguish real peaks from the background noise, which then degrades the accuracy of peak position determination and material identification. Longer exposures improve the S/N ratio by the well-known square root relationship (S/N∝√t, where t=exposure time). So, for example, a quadrupling of the exposure time doubles the S/N ratio.

High spectral resolving power in a diffraction pattern is also desirable in an EDXRD system since it controls the ability to see closely spaced peaks as a single broad peak when the resolution is poor or as two or more sharper, distinct peaks when the resolution is high. The resolving power is a convolution of the system's response and the test material response to coherent scattering. The EDXRD system contributes to diffraction peak broadening through the use of an extended X-ray source focal plane, finite collimator slit openings, and the intrinsic energy-dependent resolution of the detector. The test object itself also contributes to the diffracted peak broadening through low absorption resulting in an extended diffraction volume (VOXEL), heterogeneously distributed internal stresses, and particle size effects among others.

An example of a first generation EDXRD system is shown in FIG. 1. The X-ray source 1 is ideally represented as a point source that emits a polychromatic beam 2 over a wide angular range. A small portion of this beam passes through a narrow slit 3 in the incident beam collimator 4 to produce the desired incident beam 5. The collimator 4 has sufficient absorbing power and thickness to allow only those beams passing through the slit 3 to reach the test object 6. A portion of the transmitted beam 5 will interact with the test object 6 at some diffraction plane 7 to produce coherently scattered radiation over a wide range of angles. However, slits 8 in the receiving collimator 10 allow only those diffracted rays 9 with the desired Bragg diffraction angle 12 to pass and reach the energy discriminating detector 11. The system has rotational symmetry about the center line ℄ that extends from the center of the X-ray source to the center of the detector face, which results in the diffracted beams forming a thin wall hollow cone centered about the system axis. These “pencil beam” systems typically utilize a Si or Ge detector that is cryogenically cooled. One of the most common uses for such systems are high pressure studies in which the test sample is encased in a diamond anvil cell arranged so that the incident and diffracted beams pass through semi-transparent diamonds that are used to pressurize the test sample. Although these types of high pressure systems are found in the laboratory using a rotating anode source, they are more commonly found at synchrotron facilities.

Another example of EDXRD system is illustrated in FIG. 2. Unlike the pencil beam arrangement in FIG. 1, the incident beam arriving at the sample 17 is divergent and the diffracted beam 21 arriving at the detector 22 is convergent with respect to the central axis. As before, the X-ray source 1 emits a polychromatic beam 13 over a wide angular range. A small portion of this beam passes through a narrow annular slit 14 in the incident beam collimator 15 to produce the desired incident beam 16. The transmitted incident beam 16 has a circular cross section perpendicular to the system axis and is shaped by the annular shaped slit 14. A portion of the transmitted beam 16 will interact with the test object 17 at some diffraction plane 18 to produce coherently scattered radiation over a wide range of angles. However, the annular receiving slit 19 in the receiving collimator 20 allows only those diffracted rays 21 with the desired Bragg diffraction angle to pass and reach the energy discriminating detector 22. In this example of EDXRD system, the size of the detector 22 can be very small such that compound semiconductor detectors such as CdTe, CdZnTe, GaAs and HgI₂ among others can be used. Also, many compound semiconductor detectors are able to operate at or near room temperature, which eliminates the need for cryogenic cooling required for Si and Ge detectors.

A more recent advance in the development of EDXRD systems is illustrated in FIG. 3, where more complex collimators, slits and detector designs are used to provide tomographic XRD analysis of a three dimensional object. The incident beam collimator 24 contains a series of annular slits 23 concentric about the system axis that result in a plurality of incident beams. The receiving collimator 30 also contains multiple slits 29 that direct the diffracted beams to individual elements of a segmented detector 31 composed of a number of individual elements that are physically and electrically isolated from each other. The slits 29 in the receiving collimator 30 are strategically arranged so that the diffracted rays arriving at a given detector element come from a different plane in the object. As an example, the incident/diffracted beam combination 27 that arrives at detector element 32 comes from the diffraction plane 25. Similarly, the incident/diffracted beam combination 28 arriving at detector element 33 comes from a different diffraction plane 26. Thus, each independent detector element receives diffraction data from a different region within the object, from which a two- or three-dimensional tomographic map can be created. By increasing the number of slits in the incident and diffracted beam collimators 24 and 30, and increasing the number of elements in the detector array 31, the system can be scaled to analyze large test objects. For example, this type of system has been commercially used to detect explosives hidden within checked baggage.

Many other EDXRD systems have been designed that utilize multiple or moving X-ray sources, large two-dimensional detector arrays or enhanced collimator designs that take advantage of intensity increases through the use of polycapillaries, grazing incidence total reflection or X-ray mirrors. All of these systems however, retain the undesirable behavior where the diffracted intensity and spectral resolving power are inversely related for a given X-ray source power. Moreover, systems using grazing incidence and mirror-based collimation introduce additional limitations due to their inefficiencies at the high energies typically used in EDXRD systems. Accordingly, it would be desirable to sever the link between the diffracted intensity and the spectral resolving power in an EDXRD system without resorting to the use of high power x-ray sources. Additionally, it is highly desirable to produce a compact and low cost system.

### SUMMARY OF THE INVENTION

The problems and disadvantages of EDXRD with regard to the inverse relationship between diffracted intensity and spectral resolving power are overcome by the present low cost, compact, bench-top system. This is achieved by utilizing a larger portion of the incident X-ray beam and collecting the diffracted beam more efficiently compared to previous EDXRD systems.

More particularly, the present invention provides a compact, low cost, lab bench unit for testing pharmaceuticals or similar crystalline materials in a storage container such as a large plastic bottle or blister pack for quality control (QC) or forensic analyses. For example, counterfeit or sub-potency pharmaceuticals will produce a diffraction pattern that is different from a legitimate sample and so can be detected with a high degree of confidence. Similarly, in a production environment, XRD can be used as a QC test to verify the consistency of multiple production lots. In these applications, EDXRD is an ideal analytical tool since it can examine the contents within the storage container in situ and without disturbing the container seal.

In an aspect of the invention described herein, the present EDXRD system comprises a single, low power, stationary X-ray source that produces one or more incident X-ray beams that coherently scatter with the target material to produce a plurality of diffracted beams that are summed at the single, large area, near room temperature, energy dispersive detector. By careful design of the incident and receiving beam collimators, the system takes advantage of a significant increase in the number of irradiated voxels to produce enhanced diffracted signal strength. Since each of the multiple incident beam/diffracted beam pairs has the same Bragg angle, summation of all the pairs in the single detector does not degrade the system's spectral resolving power. Accordingly, the link between intensity and resolution is severed in the present system, thus permitting a more flexible design than previous EDXRD systems.

The incident collimator slits are arranged to produce one or more concentric annular rings of X-rays by means of narrow annular slits that make a small angle with respect to the system's symmetry axis. This angle increases with radial distance from the symmetry axis and the slit opening is chosen to limit the angular divergence of the beam passed thru the slits in a radial direction. In a similar way, the receiving collimators that determine the paths of the diffracted beams also have multiple narrow annular slits that make positive, negative or no angle with respect to the system axis that extends from the center of the X-ray source to the center of the detector face. Also, the radial angular divergence of the beams passing through the receiving slits is limited by the size of the slit opening. Thus, the combined set of collimators can be arranged in a specific design to produce a pair of incident and diffracted beams intersecting at a single voxel within the test sample with high spectral resolving power. However, the diffracted spectrum from a single pair of such beams will have low intensity. But when added to other pairs with the same Bragg angle, the multiple diffracted beams arriving at the detector will be summed to produce a high intensity spectrum with unaltered resolving power.

Further enhancements in the spectral resolving power of the system can be achieved by limiting the divergence of the incident and diffracted beams tangential to the annular ring of each slit in each of the collimators. These tangential collimators can be either discrete components centered on the system axis or they may be combined with the radial collimators in a more complex, monolithic body produced by additive manufacturing methods. This greatly simplifies system alignment and reduces the system size, complexity and cost.

In one aspect, by using a low-power X-ray tube, radial and tangential collimators, and a large area, near room temperature detector, the present system is capable of examining bottles or containers housing pharmaceuticals with a goal of detecting counterfeit or sub-potency materials. In a further aspect, the use of a plurality of incident X-ray beams permit the inspection of blister packs containing a sparse population of pills, capsules, caplets, lozenges and so forth to detect counterfeits or sub-potency materials. In another aspect, the system can be used for quality control inspection of production materials to verify lot-to-lot uniformity. In a further aspect, the system can be used to detect polymorphic transformations in production lots of pharmaceuticals and other crystalline materials that may have undergone phase transformation during processing.

In another aspect of the invention, tools have been developed to further enhance the EDXRD system. One such tool is an algorithm to determine the optimal Bragg diffraction angle in the EDXRD system for a particular drug, a family of drugs, chemicals or other crystalline materials based on the known ADXRD patterns that are widely available. A second tool is an algorithm for designing the collimator positions and the associated slit positions and their related angles with respect to the system axis. A third tool is the development of an algorithm to assess the performance of a particular EDXRD system and its sensitivity to alignment and manufacturing errors.

The features of the invention are exemplified by the following drawings and detailed description.

### BRIEF DESCRIPTION OF THE DRAWINGS

FIG. 1 illustrates a pencil beam EDXRD system.

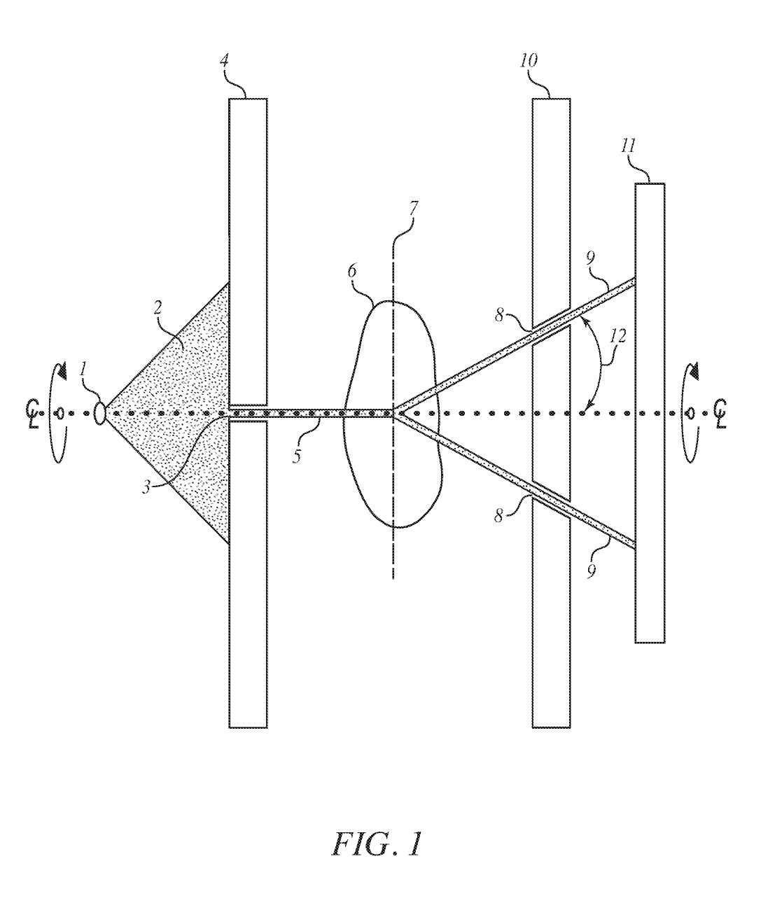

FIG. 2 illustrates a cone-based EDXRD system.

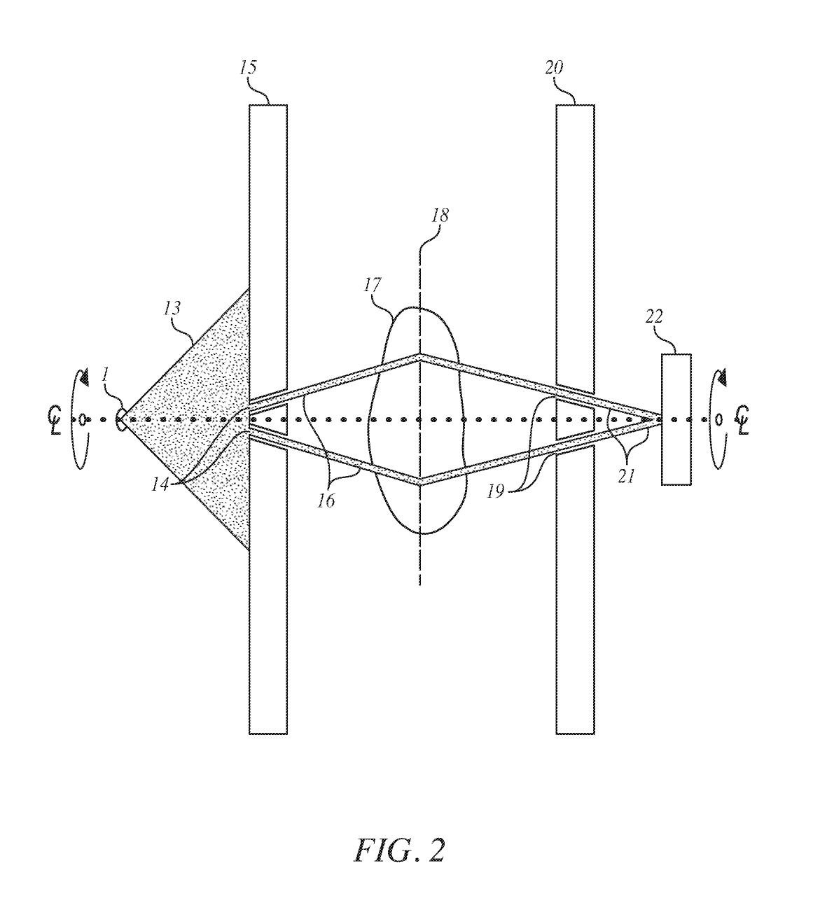

FIG. 3 illustrates a tomographic EDXRD system based on the use of large detector arrays.

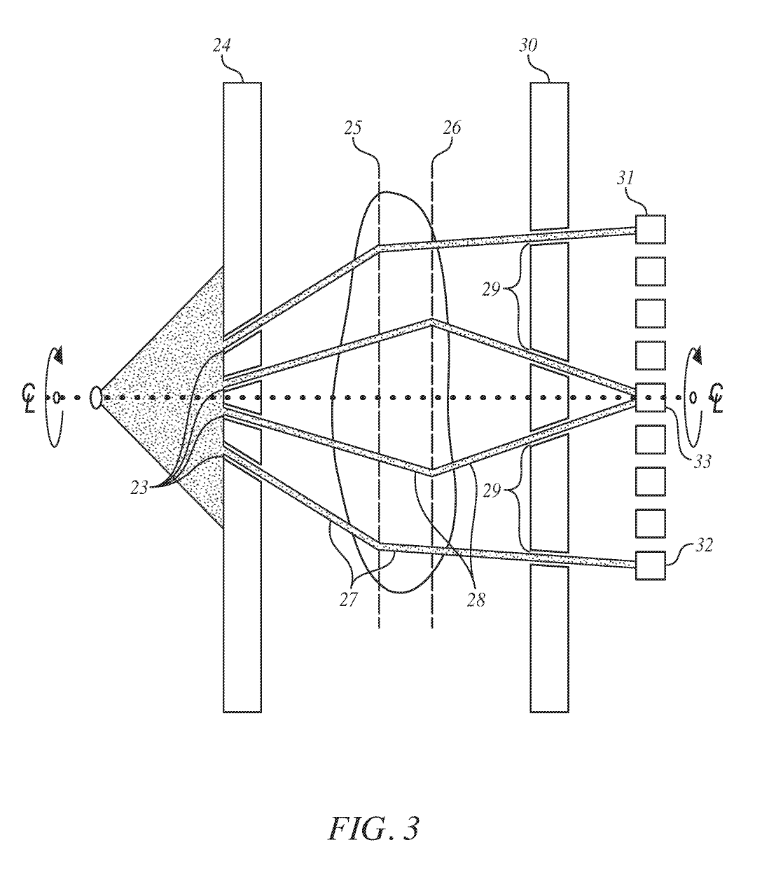

FIG. 4 is a longitudinal view of a compact, low cost EDXRD system according to the present invention that extends from the center of the X-ray source to the center of the detector face.

FIG. 5 is a transverse view of the incident beam collimator.

FIG. 6 is an enlarged view showing the Bragg angle formed by each incident beam and the corresponding diffracted beam relative to the system axis that extends from the center of the X-ray source to the center of the detector face.

FIG. 7 is a transverse view of an incident X-ray beam showing the radial and tangential divergences after passing through a slit in the incident beam collimator.

FIG. 8 is a transverse crossectional view of a wagonwheel collimator to limit tangential divergence.

FIG. 9A shows the transverse view of a monolithic hybrid collimator combining both receiving collimators of FIG. 4 and the tangential collimator of FIG. 8. FIG. 9B shows the longitudinal view of a monolithic hybrid collimator combining both receiving collimators of FIG. 4 and the tangential collimator of FIG. 8.

FIG. 10 is a longitudinal view of a compact, low cost EDXRD system according to the present invention with the system axis extending from the center of the X-ray source to the center of the detector face.

FIG. 11 is a longitudinal view of a compact, low-cost EDXRD system according to the present invention with the system axis extending from the center of the X-ray source to the center of the detector face.

FIG. 12 is a longitudinal view of a compact, low-cost EDXRD system according to the present invention with the system axis extending from the center of the large X-ray source to the center of the detector face.

FIG. 13 is a longitudinal view of a compact, low-cost EDXRD system according to the present invention with the system axis extending from the center of the X-ray source to the center of the detector face.

FIG. 14A is a longitudinal view showing an original hourglass slit and FIG. 14B is a longitudinal view showing a new chevron slit after translation illustrating how shifting the receiving collimator reduces some of the design and manufacturing complexity.

FIG. 15A is a view along the system axis showing the projection of a single X-ray incident annular ring on a blister pack containing pharmaceuticals. FIG. 15B is a view along the system axis showing the projection of multiple X-ray incident annular rings on a blister pack containing pharmaceuticals.

FIG. 16 is a flowchart showing the algorithm to computationally determine the optimal Bragg angle for an EDXRD apparatus.

FIG. 17 is a diagram showing some parameters of the system of the present invention

FIG. 18 is a block diagram showing the functions of the Monte Carlo program to evaluate the performance of an EDXRD system.

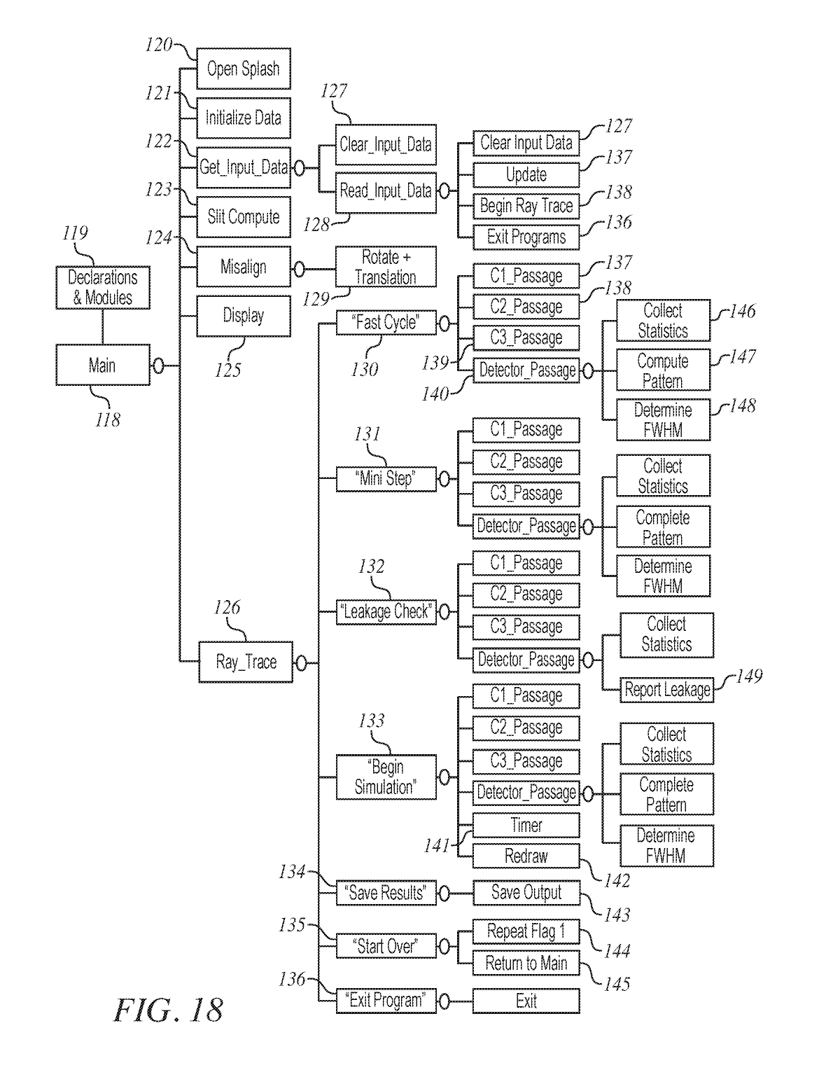

FIG. 19 shows the angle of acceptance for a slit in the incident beam collimator containing a misalignment

FIG. 20 shows the interaction of the incident beam with the discretized sample element.

FIG. 21 shows the angle of acceptance for a slit in the diffracted beam collimator containing a misalignment.

FIG. 22 shows the diffraction voxel and the diffraction plane defined for multiple voxels with the system axis extending from the center of the X-ray source to the center of the detector face.

FIG. 23 shows an expanded view of the diffraction voxel relative to the diffraction plane defined by the intersection of the incident and diffracted beams.

### DETAILED DESCRIPTION OF THE INVENTION

Other than in the operating examples, or where otherwise indicated, all numbers expressing quantities, energy range, thermal conditions, and so forth, used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, inclusive of the recited values, may be used.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

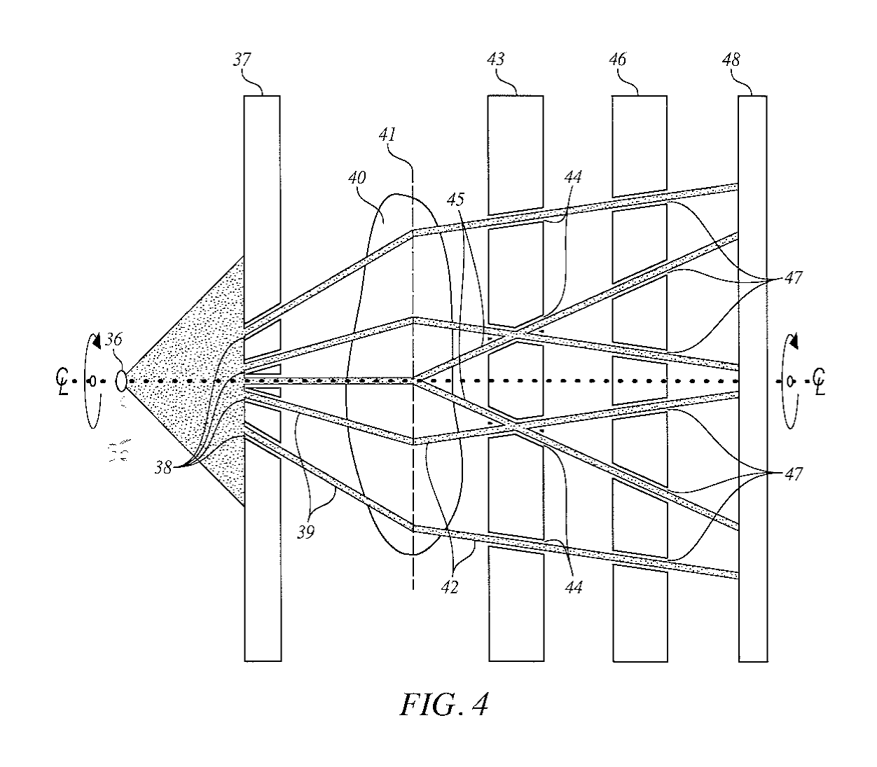

FIG. 4 is a longitudinal cross-sectional view of an EDXRD system along the system's symmetry axis defined by a line extending from the center of the X-ray source to the center of the detector face and comprises an X-ray source 36, an incident beam collimator 37 containing a plurality of non-parallel annular slits 38, two diffracted beam collimators 43 and 46, each containing a plurality of non-parallel annular slits 44 and 47, respectively and a single energy dispersive detector 48. The incident beam collimator 37, the diffracted beam collimators 43 and 46, and the detector 48 are perpendicular to the system axis. The system has rotational symmetry about the system axis ℄ such that each of the various slits form an annular ring as illustrated in

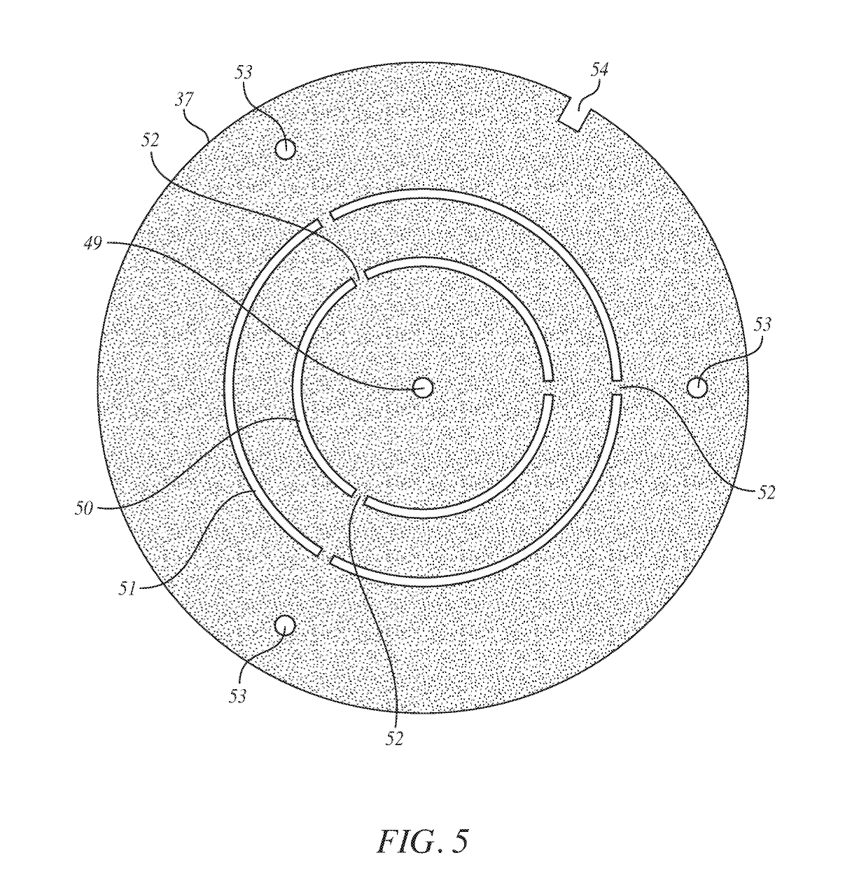

FIG. 5, which is a transverse view of the incident beam collimator 37. In this transverse view of the incident beam collimator 37, the system axis is perpendicular to the collimator face shown and passes through the center slit 49. For simplicity, the incident beam collimator 37 shown in FIG. 4 and FIG. 5 shows only three annular slits, but one skilled in the art will recognize that additional annular slits can be added to further improve the system performance. As shown in FIG. 5, the annular slits 49, 50 and 51 are concentric with the system symmetry axis. Each of these annular slits will have a different angle with respect to the system axis such that each annular slit has the X-ray source 36 at its apex 35. In order to support each annular slit, support legs 52 must be used, which will slightly reduce the incident beam intensity. Alignment slots 53 are also included to attach the collimator to an external fixture and adjust the pitch and yaw, while slot 54 sets the in-plane rotation angle.

The alignment slots 53 may be used in a variety of ways to hold the collimator 37 and align it with respect to the system axis. A preferred embodiment is to use bolts passing through the alignment slits 53 to mechanically attach the collimator 37 to one or more gimbals that correct for pitch or yaw errors. Alternatively, bolts passing through the alignment slits 53 may be used to attach the collimator 37 to a rigid frame that is well aligned to the system axis. A third embodiment is to press fit the collimator 37 into a cylindrical tube containing a longitudinal keyway and with an inside cylinder diameter that closely matches the outer diameter of the collimator 37. A key is then pressed into the collimator keyway 54 and the mating keyway in the cylinder wall to prevent in-plane rotation of the collimator 37. One skilled in the art will recognize that similar methods can be used to attach and align the diffracted beam collimators 43 and 46.

The ideal material of construction for all of the collimators 37, 43 and 46 is tungsten or its alloys such as machinable tungsten/(Cu, Ni) or tungsten carbide, although other heavy metals may also be used. Alternate materials such as the common transition metals and their alloys are also suitable. Tungsten and its alloys will produce a strong fluorescence line in the diffraction pattern at approximately 59.3 keV, while the transition metals such as Cr, Mn, Fe, Co, Ni, Cu and Zn (found in steels, Inconels and brass among others) will introduce fluorescence lines below 8.65 keV or less. One skilled in the art of EDXRD will recognize that fluorescence lines at these energies (<8.65 keV or 59.3 keV) will be outside the region of interest for EDXRD pattern analysis, which is typically 10-60 keV. Accordingly, any of the cited materials can be used to manufacture the collimators. But whatever material is used, it must be sufficiently thick to limit spurious radiation leakage through the collimator wall.

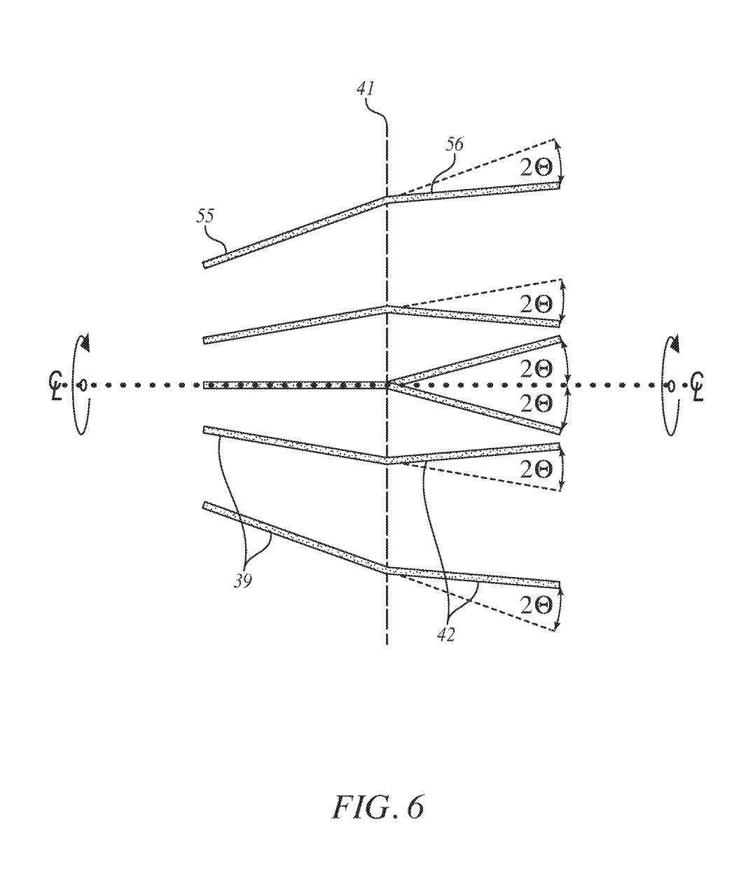

Referring to FIG. 4 and FIG. 5, the X-ray source 36 emits a cone of polychromatic X-rays that is symmetric about the system axis previously defined over a broad angular range. The incident beam collimator 37 has sufficient mass absorption coefficient and thickness to block all of this radiation, except those few rays that are aligned with the collimator annular slits 38. The collimator 37 permits two annular rings of radiation 39 to pass thru slits, 50 and 51 in addition to a pencil beam that passes through the center hole 49. These incident X-ray beams penetrate the test object 40 and a portion of each incident ray will result in coherent scattering. The receiving collimators 43 and 46 are designed so that the only diffracted beams that can reach the detector 48 originate from the same plane 41 perpendicular to the system axis in the test object. Also, each diffracted beam originating at plane 41 and passing thru the slits 44 in collimator 43 and slits 47 in collimator 46 all have the same 20 Bragg angle 42 as illustrated in FIG. 6. The incident X-ray beam and the resulting diffracted beam form a diffraction pair such as that shown by 55 and 56 and originating at plane 41. By design of the incident and receiving collimators 37, 43 and 46, all five diffraction pairs illustrated in FIG. 6 have the same 2Θ Bragg angle required for diffraction. For simplicity, only a few such diffraction pairs are shown, but one skilled in the art will recognize that additional pairs can be added to further enhance the system's performance.

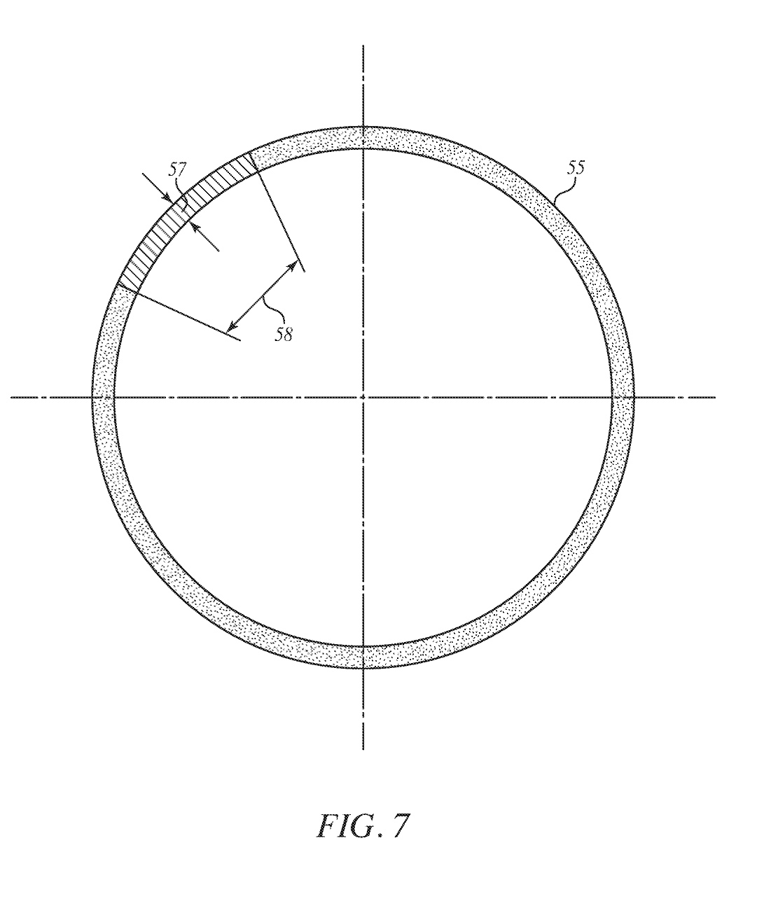

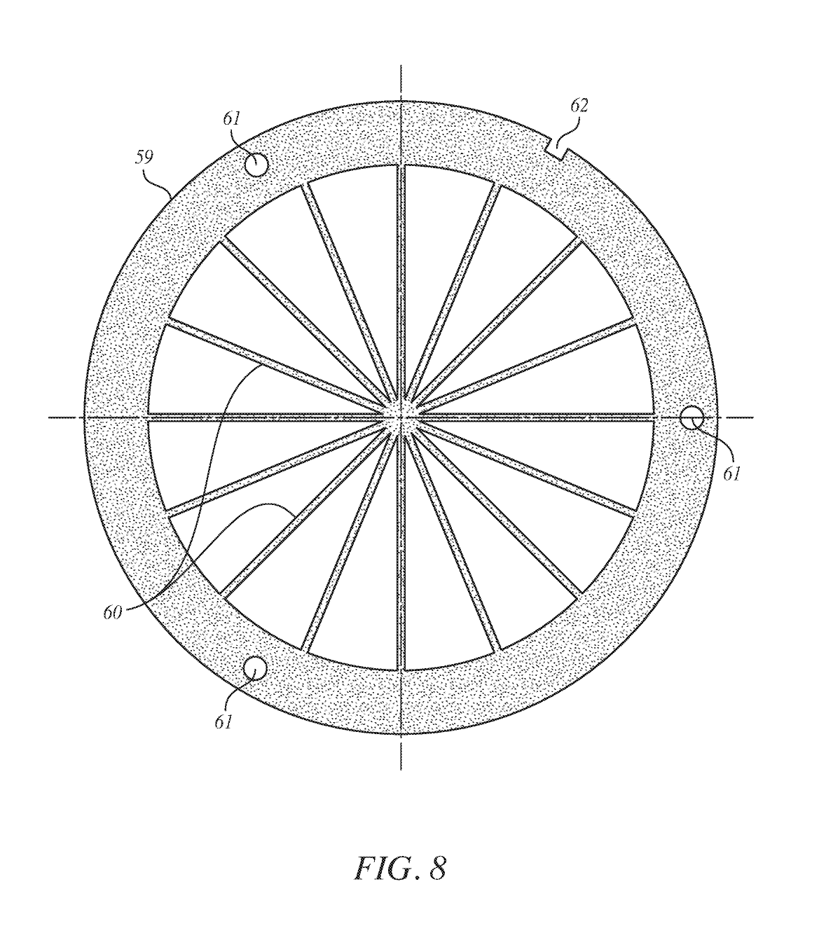

The X-ray beams exiting the incident beam collimator 37 and the first receiving collimator 43 have both a radial and tangential divergence, which will contribute to the broadening of the diffraction peaks, thus degrading the system spectral resolution. The radial divergence is defined as the angular spread of the X-ray beam perpendicular to the system axis and is controlled by the slit opening in collimator 37, 43 or 46. Tangential divergence, on the other hand, is defined as the angular spread of the X-ray beam at a constant radius from the system axis. FIG. 7 shows a transverse view of the incident X-ray beam 55 after passing through one of the slits in collimator 37. The radial divergence 57 is limited by the slit width while the tangential divergence 58 is only partly limited by the slitwidth, which must be further augmented by other means such as a secondary tangential collimator. FIG. 8 is a transverse view of a collimator 59 to limit the tangential divergence and consists of a series of thin plates 60 in a wagonwheel arrangement such that all of the plates are evenly distributed about the system's symmetry axis, which passes through the center of the collimator. The preferred embodiment of the tangential collimator 59 comprises a single monolithic structure that can be manufactured by powder metallurgy, 3-D printing or electro-discharge machining. The tangential collimator 59 also contains alignment holes 61 and keyway 62 to accommodate locating pins or key that fit the alignment holes 53 and keyseat 54 in the radial collimator 37, 43 or 46 to which it is attached. Although the tangential collimator 59 need not be physically attached to the receiving collimators 43 or 46, it is preferred that both receiving collimators 43 and 46 be attached to form a rigid and well aligned unibody. A preferred embodiment comprises bolts that align the alignment slits in the first diffracted beam collimator 43 to the mating alignment holes 61 in the tangential collimator 59. In a similar way, the second diffracted beam collimator 46 is attached to the exit end of the tangential collimator 59 with bolts passing thru the alignment holes in the second diffracted beam collimator and aligning with the corresponding alignment holes 61. An additional method comprises sliding the diffracted beam collimators 43 and 46 on either end of the tangential collimator 59 into a close fitting cylindrical housing into which a longitudinal keyway has been machined and aligning the three collimators by means of a key pressed into the keyway 62 and the corresponding keyways in the diffracted beam collimators 43 and 46 and the cylindrical housing.

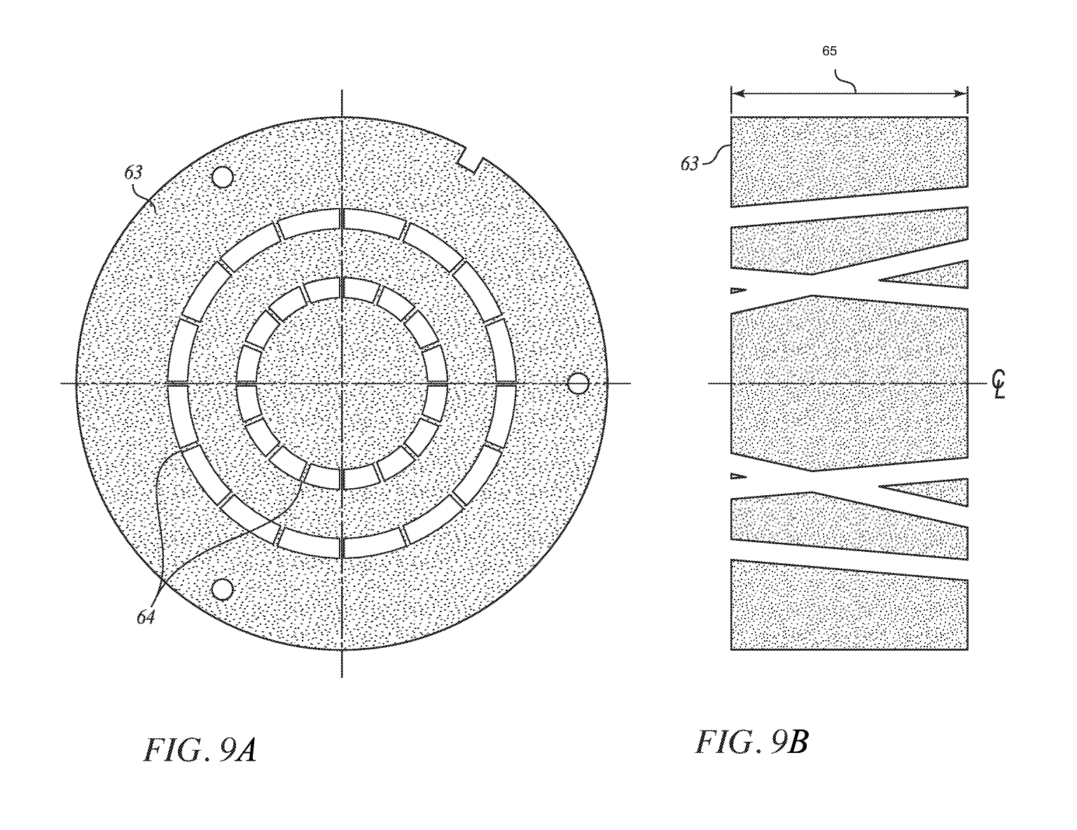

The tangential collimator can be combined with the radial collimators 43 and 46 with the tangential collimator 59 to form a single, monolithic structure 63 as illustrated in FIG. 9. The thickness 65 of the structure in the longitudinal direction parallel to the system symmetry axis is equivalent to the combined thickness of the two receiving collimators 43 and 46 plus the separation distance between them. By creating a hybrid combination 63 of the three collimators, 43, 46 and 59, some of the alignment slits can be eliminated, misalignment errors are greatly reduced, and several support structures to hold the individual components can be eliminated, thus reducing the system's size, complexity and cost. The preferred embodiment for constructing the hybrid collimator 63 comprises the use of 3-D additive manufacturing methods, which build the structure in three dimensions by depositing the desired metallic powders layer by layer.

Materials of construction for the hybrid collimator 63 include tungsten, machinable tungsten alloys or tungsten carbide. However, the size and complexity of the collimator combined with the inherent difficulties of machining tungsten and its alloys and compounds will require special manufacturing methods. Conventional machining, such as subtractive manufacturing using CNC (Computer Numerical Control) or ESD (Electro Spark Discharge) methods, for example, will be very difficult to use. Fortunately, additive 3-D manufacturing methods have matured and are now capable of producing the desired hybrid collimator by methods including, but not limited to, Powder Bed Laser Melting, Direct Metal Laser Melting, Selective Laser Melting, Laser Deposition and Electron Beam Melting. These methods can also be used to produce the hybrid collimator 63 from the common transition metal based alloys such as, but not limited to, stainless steels, nickel-based alloys, brasses, bronzes and similar materials. These materials of construction and methods of construction apply to all of the incident and receiving collimators described in the present invention.

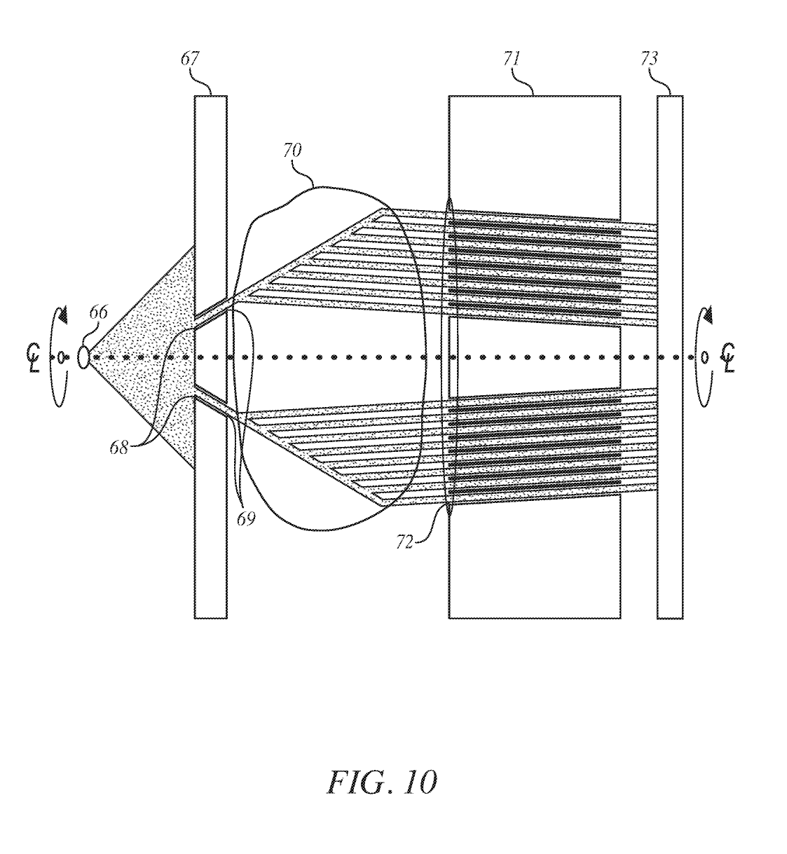

A further aspect of the present invention includes the EDXRD system illustrated in the longitudinal cross-sectional view of FIG. 10 comprising an X-ray point source 66, an incident beam collimator 67 with a single annular slit 68 centered on the system symmetry axis, which is the line extending from the center of the X-ray source to the center of the detector face. The collimator 67 has sufficient thickness to absorb all the incident X-rays except those passing through the annular slit and producing the annular incident beam 69. Portions of the annular beam 69 interact with the test object 70 to produce diffracted beams originating from numerous points along the incident beam path that are capable of passing through the series of parallel slits 72 in the thick receiving collimator 71 before reaching the near room temperature energy dispersive detector 73. The annular slit in the incident beam collimator 67 is placed at a distance from the X-ray source 66 such that the slit's apex coincides with the X-ray source point focus. The receiving collimator 71 is a thick, monolithic body into which a series of concentric annular slits are arranged such that all of the transmitted beams are parallel to each other. Thus, the Bragg angles of all diffracted beams reaching the detector 73 have the same nominal value with a resultant improvement in the intensity of the diffracted intensity without degradation of the system's spectral resolution.

The receiving collimator can be manufactured as three separate components—a first collimator limiting the radial divergence, a second collimator further limiting the radial divergence and defining unique beam paths, and an optional, intervening collimator to limit the tangential divergence as previously described.

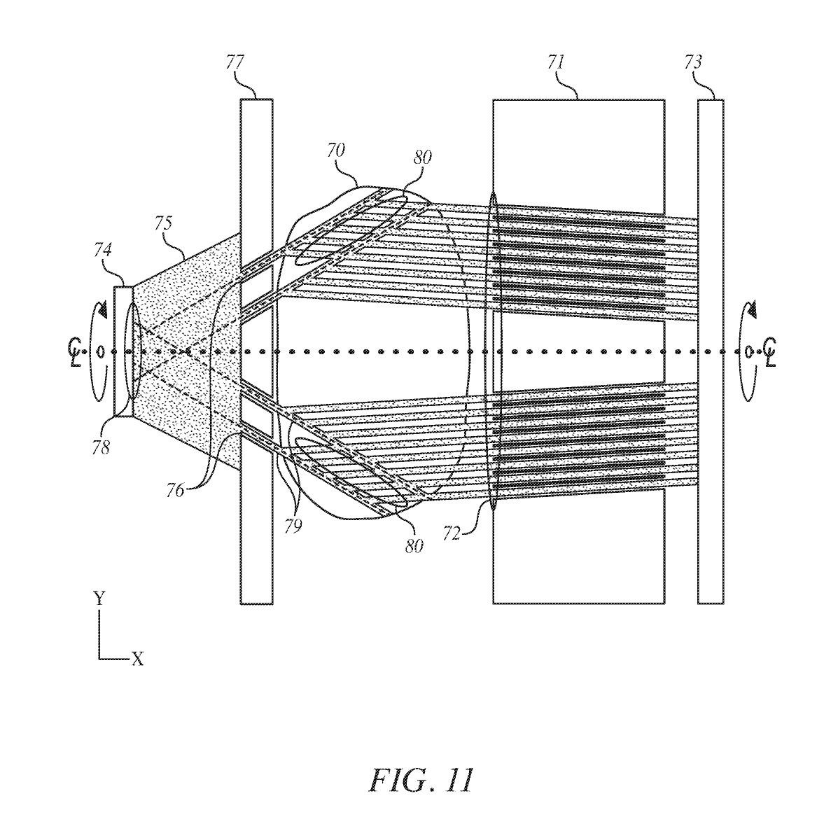

A further aspect of the present invention includes the EDXRD system illustrated in a longitudinal cross-sectional view of FIG. 11, where the X-ray point source of the previous examples, viz. 1, 36 and 66 is now replaced by an extended source 74 in the YZ plane with dimensions of several millimeters in each direction. A large X-ray focal plane increases the number of potential parallel incident beams that can be created and successfully pass through the incident collimator 77. Thus, X-rays generated at multiple points 78 in the X-ray tube focal plane are able to pass through the parallel and concentric annular slits 76 in the incident beam collimator 77 to produce two or more parallel incident X-ray beams 79 that interact with the test object 70. Each of the annular incident beams produces a number of diffracted beams that can pass through the multiple slits in the receiving collimator 71, which is identical in design and limitations to the one illustrated in FIG. 10. By use of the multiple incident X-ray beams, additional diffracted rays 80 are produced over that produced by the system illustrated in FIG. 10. One of the considerations in the arrangement of this example is that the multiple annular slits 76 no longer have a common apex located at the X-ray tube focal point 66 illustrated in FIG. 10. Instead, the angular opening of each annular ring in collimator 77 is the same and the apex for each ring lies at a different point along the X-ray source plane 74 and may lie below or above the system symmetry axis.

For simplicity, FIG. 11 shows only two concentric annular rings of incident X-rays arriving at the object, but one skilled in the art will recognize that many additional parallel incident beam paths can be created, limited only by the size of the extended X-ray source 74. In comparison to the system illustrated in FIG. 10, the example shown in FIG. 11 produces additional diffracted beams 80, all of which are parallel, thus increasing the intensity of the diffracted beam without sacrificing the overall system resolution.

Spot sizes for low power X-ray tubes are typically in the range of 0.05 mm to 0.15 mm, while X-ray tubes with larger focal spots are readily available in the 1.0 to 8.0 mm range. Thus, shifting from a standard small focus tube with 0.15 mm focal spot to the much larger 8.0 mm focal spot could produce an incident beam array with an intensity increase of an order of magnitude or more by utilizing the design of the incident collimator 77 of FIG. 11.

The EDXRD systems illustrated by FIG. 10 and FIG. 11 are most effective when the test object is large and homogeneous, where a large number of voxels within the object space are available for coherent scattering. One potential application contemplated for the present invention is the interrogation of large containers of pharmaceuticals for forensic or quality control analysis. In this case, large cylindrical bottles up to 20 cm diameter or more and contain hundreds to thousands of pills, tablets, capsules, etc. (hereafter referred to simply as pills), fulfill this requirement for a large and homogeneous test object. Although there are vacant spaces between each of the pills, these interstices are filled with air or other protective gasses, neither of which scatter X-rays coherently. Thus, the only contributions to the total X-ray diffracted pattern come from the pharmaceuticals and their excipients and not from the material in the voids between the pills.

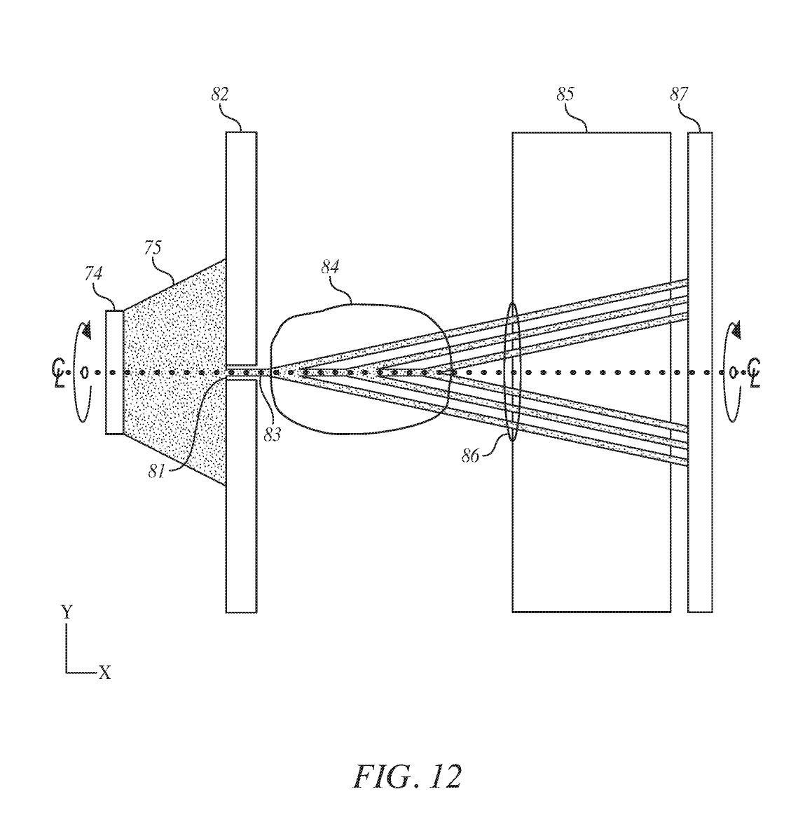

A further aspect of the present invention includes the EDXRD system illustrated in a longitudinal cross-sectional view of FIG. 12, which is most applicable to test objects that are much smaller than that utilized in the example illustrated in FIG. 11. The X-ray source 74 may be either a point or extended source as previously described, which produces a fan beam 75 over a broad angular range. A pinhole slit 81 in the incident beam collimator 82 produces a single pencil beam 83 along the system's symmetry axis that interacts with the small sample 84. The receiving collimator 85 comprises a thick, monolithic structure with two or more parallel annular slits 86 that allow diffracted beams from a plurality of originating voxels within the sample along the system's symmetry axis to reach the detector 87. The receiving collimator may be either of the discrete type (two receiving slits with an optional tangential collimator as described previously) or of the monolithic type where all of the functions are combined into a single component manufactured by 3-D printing methods. The advantage of this example is that extraction of diffracted beams from multiple voxels enhances the diffracted intensity without degrading the system's spectral resolution.

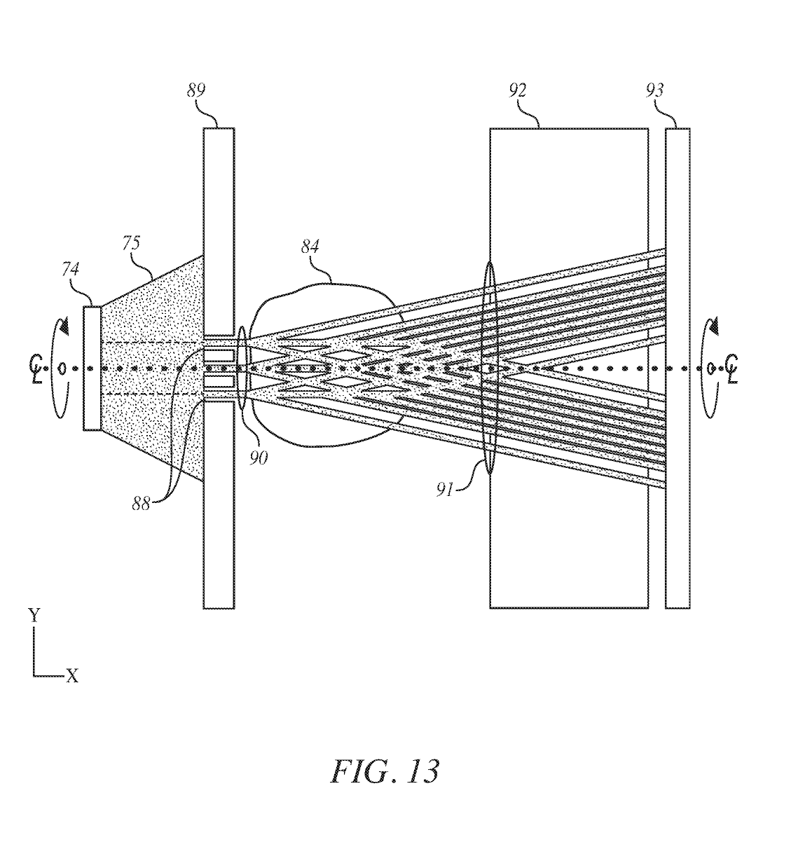

A further aspect of the present invention includes the EDXRD system illustrated in a longitudinal cross-sectional view of FIG. 13, which is most applicable to test objects that are intermediate in size between the small sample of FIG. 12 and the very large sample of FIG. 11. The X-ray source 74 is an extended source as previously described which produces a fan beam 75 over a broad angular range. A plurality of pinhole slits 88 in the incident beam collimator 89 produces two or more pencil beams 90 along the system's symmetry axis that interacts with the intermediate sized sample 84. The multiple incident beams may lie on the system's symmetry axis or may be displaced from the axis, but all of the beams are parallel to each other and to the system axis. The receiving collimator 92 comprises a thick, monolithic structure with a plurality of parallel annular slits 91 that allow diffracted beams from the numerous originating voxels within the sample to reach the detector 93. As with the previous examples, the receiving collimator may be either of the discrete type (two receiving slits with an optional tangential collimator) or of the monolithic type where all of the functions are combined into a single manufactured component. Since all of the incident beams are parallel to each other and the annular rings of diffracted beams are parallel to each other, the Bragg angles and their divergences for all beam pairs are the same. Thus, the plurality of diffracted beams arriving at the detector 93, which are then summed, will not degrade the system's resolving power, but will increase the intensity of the diffracted pattern.

The number of incident beams 90 is limited by the size of the X-ray source 74 and also by the size of the sample under test. Similarly, the number of parallel diffracted beams that can be utilized is also limited by the sample size. In addition, the maximum size of the detector that is available will limit the number of diffracted beams that can be collected.

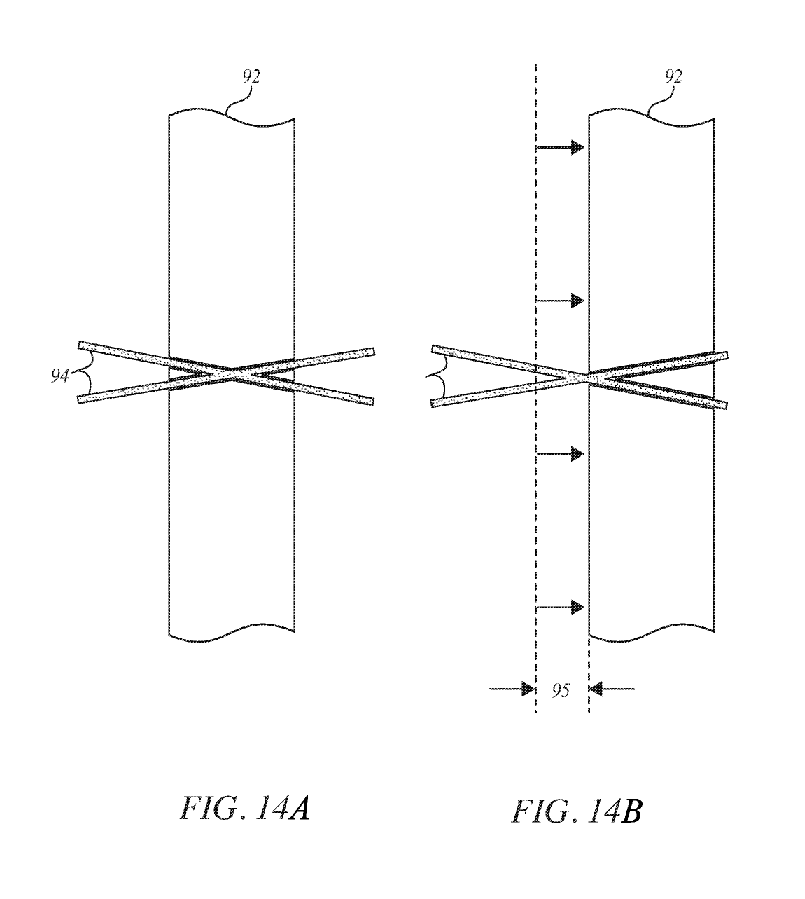

In the system illustrated in FIG. 13, a few of the diffracted beams cross each other's path, which may present manufacturing challenges due to lack of mechanical support of some of the smaller regions, which appear as floating islands in the longitudinal view. One option to avoid these problems (see FIG. 14) is to translate the collimator 92 further away from the test sample by a small amount 95 such that the original hourglass shaped slit required by the crossing diffracted beams 94 can be converted to a chevron shaped slit that is easier to manufacture but which then requires the use of a slightly larger detector. A compromise is to simply abandon one or more of those few diffracted beams that cross, which decreases the diffracted intensity slightly, but retains the advantage of using a smaller detector.

The EDXRD systems described in the examples described above enable the development of a compact, low-cost apparatus by more efficient use of the X-ray source or more efficient collection of the coherently scattered diffracted X-ray beams or a combination thereof. Specifically, the increased diffracted intensity that results from the collimator designs of the present invention and use of X-ray tubes with a large focal spot, greatly reduce the size and power requirements for the X-ray source. As a result, high diffracted intensities can be achieved with self-contained air-cooled or oil-cooled X-ray sources that require no external cooling media. Power requirements for such X-ray sources are typically in the range of 10 W to 150 W, which greatly reduces the size, weight, complexity, shielding and cost of the apparatus. The size, weight, complexity and cost are also reduced by the use of near room temperature detectors such as CdTe, CdZnTe, HgI₂, GaAs or similar compound semiconductors with high stopping power. Since these detectors operate at or near room temperature, only simple cooling of the detector is required, such as that provided by an on-chip Peltier device to provide sufficient cooling for adequate detector response. In the present invention, the detectors should be as large as possible, preferably of the order of 25 mm diameter, in a single monolithic device so that the plurality of diffracted beams is summed automatically without the need for external electrical circuits to accomplish the summation. A system and/or apparatus comprised of the air/oil cooled, low power X-ray source, multi-slit incident and receiving collimators, a large area, near room temperature detector along with the associated power supplies, control electronics, detector chain, radiation shielding and computer can now be constructed that fits on a laboratory bench and weighs only a few hundred kgs. By contrast, conventional EDXRD systems used for baggage inspection are floor mounted systems occupying approximately 15 m² or more and weigh approximately 10,000 kg.

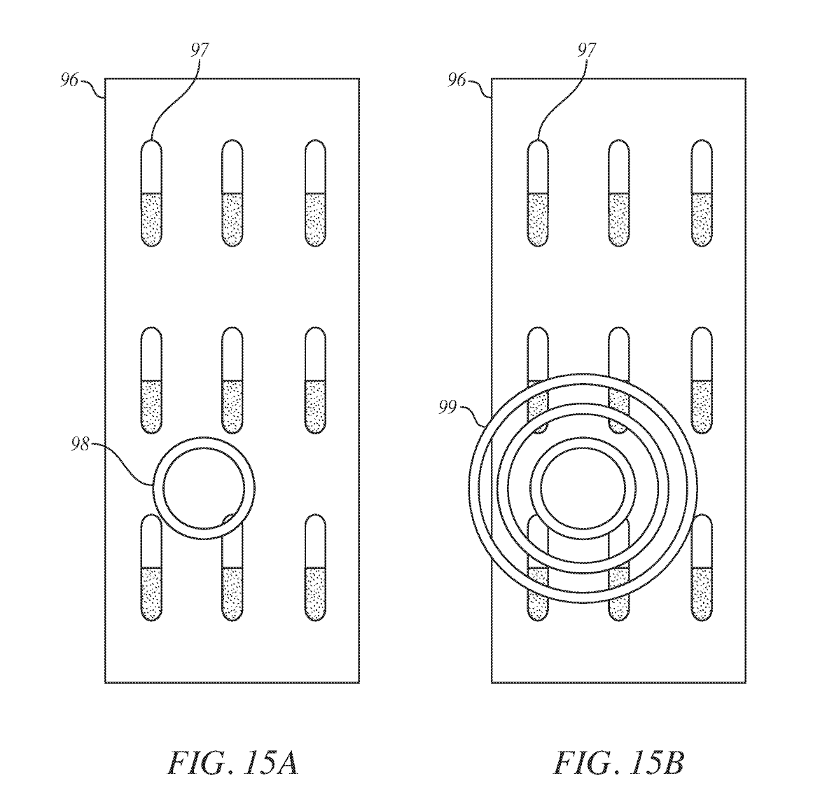

Applications envisioned for the present invention are those where the test samples are small, such as bottles commonly used to contain hundreds to thousands of pharmaceutical pills, tablets, capsules, caplets, films, pastes, powders, pastilles, lollipops, lozenges, troches, gums and so forth. Such pharmaceuticals are tested for legitimacy or subpotency by comparing the diffraction pattern from a test sample to that taken from a standard sample collected under identical test conditions. Methods for analyzing such diffraction patterns may include simple table lookup procedures involving peak position matching, or more advanced methods such as neural networks, neural tree networks, Fourier, correlation and cepstral methods among others, which are well known to those skilled in the art and are often used for pattern recognition. The present invention may be used for quality control (QC) validation of production lots of pharmaceuticals by monitoring the diffraction patterns on a continuous basis, whereby changes in the diffraction patterns can indicate small drifts in production methods and can be determined with ease. The system of the present invention may also be used in the detection of polymorphic transformations of a pharmaceutical, which may affect a drug's bioactivity and may occur in pharmaceuticals due to temperature or humidity conditions, or to intense shear during mixing. This sort of change typically cannot be detected by chemical methods, since all polymorphs of the same drug have the same chemical composition. The present invention may also be used in the examination of blister packs, especially when the system illustrated in FIG. 11 is used. As shown in FIG. 15a, an EDXRD system using only a single annular incident beam 98 is likely to miss the pharmaceutical 97 that is sparsely distributed on the blister pack backing card 96. But when the multiple annular incident beam arrangement 99 is used, as in FIG. 15b, the pharmaceutical 97 is much more likely to be irradiated.

Pharmaceuticals of the present invention may include one or more active substances. Such active substances may be any known active pharmaceutical ingredients such as, inter alia, antacids, antiallergics, analgesics, hormones, steroids oestrogens, contraceptives, nasal decongestants, H₁ and H₂ antagonists, β₂ stimulants, vasodilators, antihypertensives, anti-infective agents, laxatives, antitussives, bronchodilators, agents against sore throats, bismuth and its salts, fungicides, antibiotics, stimulants (such as, for example, amphetamines) alkaloids, oral hypogylcaemics, diuretics, cholesterol-lowering agents, combinations of various pharmaceutically active agents, and so forth. It is also possible to use the system of the present invention in the detection of pharmaceuticals comprising one or more active substances. Applications of the present invention are not limited to pharmaceuticals, but also to other crystalline materials such as, but not limited to inorganic and organic chemicals, minerals, cements, metals, cosmetics, bioceramics, excipients, ferroelectrics, explosives, ionic conductors, pigments, superconductors, zeolites, abrasives, composites, semiconductors, intercalates, hazmats, metamaterials, thermoelectrics, piezoelectrics, pyroelectrics, photonics, radionuclides, dyes, tailings, slags, clays, silts, enzymes, proteins, vitamins and polymers.

### System Design Tools

The most important parameter for designing an EDXRD system is the choice of Bragg angle 12 & 42. If this angle is set too high, the EDXRD diffraction pattern shifts to low energies where the diffracted beams are subject to self-absorption, making identification difficult. But if the Bragg angle is set too low, the diffraction pattern shifts to very high energies resulting in a pattern with very few peaks, all with low intensities. Thus, the Bragg angle should be set at an optimal intermediate value to maximize the number of strong diffraction lines within the energy region of interest, which past experience has shown to be approximately 10 keV to 60 keV.

Further complicating the problem of setting the optimal Bragg angle for an EDXRD system is the fact that the angle will vary for different classes of materials. For example, organic materials typically have large crystal d spacings (used in eq. 1), which require a low Bragg angle setting for an optimally designed EDXRD system. By contrast, inorganic materials typically have smaller crystal d spacings, which require a higher EDXRD Bragg angle. Thus, an EDXRD system designed for testing pharmaceuticals or other organic materials would not function well for testing metals or most inorganics.

The optimal Bragg angle for a given test material can be determined experimentally, but this is expensive, time consuming and cumbersome since a new set of collimating slits in systems such as those described previously must be manufactured for each Bragg angle. Accordingly, it is highly desirable to have an alternate means of determining the optimal Bragg angle for the intended application. The present invention accomplishes this goal with a computational tool that converts ADXRD patterns of the test material to an equivalent EDXRD pattern with for a particular Bragg angle setting, X-ray source characteristics and the instrument response. Repeated use of such a tool with different Bragg angles would then reveal which angle is optimal.

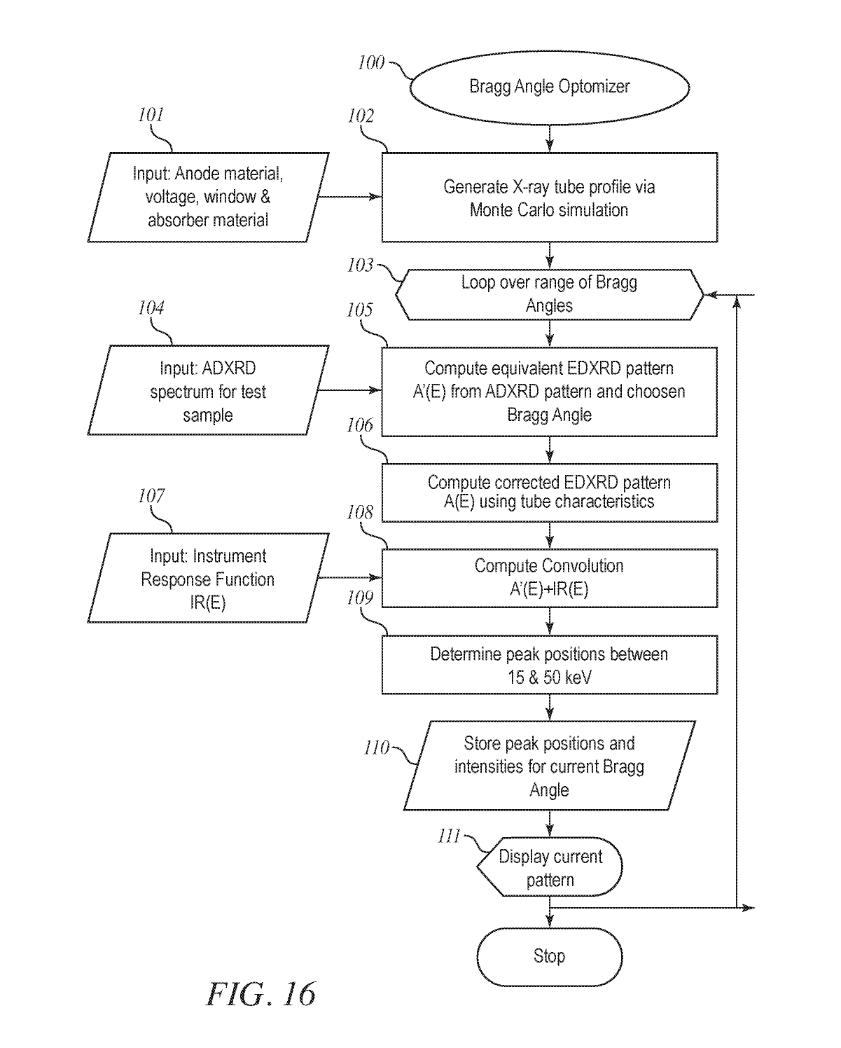

FIG. 16 is a flowchart of an algorithm 100 for converting ADXRD patterns to an equivalent EDXRD patterns. The first step 102 is to compute the spectral response of the X-ray tube based on the Monte Carlo method, using input data 101 such as the excitation potential, the anode, window and absorber materials. A loop 103 is then initiated with a range of Bragg angles. For each Bragg angle, the ADXRD data 104 are converted into the equivalent EDXRD data. The experimental ADXRD patterns are simple to obtain or can be calculated from first principles using crystallographic data contained in several commercial databases. For example, the Powder Diffraction File (PDF) contains several hundred thousand experimental patterns of both organic and inorganic materials. Other databases such as the Cambridge Structural Database (CSD), the Inorganic Crystal Structure Database (ICSD), the Pauling File, CRYSTmet, Crystallographic Open Database (COD), the Mineralogy Database and RCSB Protein Data Bank, among others, archive atomic coordinates for crystalline materials, which can be used to compute an ADXRD pattern. In toto, all of the databases catalog structural data for more than 1,000,000 materials. After conversion into the EDXRD pattern 105, the peak intensities are corrected 106 to account for X-ray source intensity profile 102. The resulting profile then undergoes a convolution with the Instrument Response Profile 108 using either an experimental or calculated profile 107 that describes the effect of the slits, x-ray source and detector on the line broadening. Finally, for each spectrum thus obtained, all observable peaks are identified 109 and quantified with respect to their energy and intensity 110. The optimal Bragg angle is then selected according to which angle provides the greatest number of observable peaks over the energy region of interest.

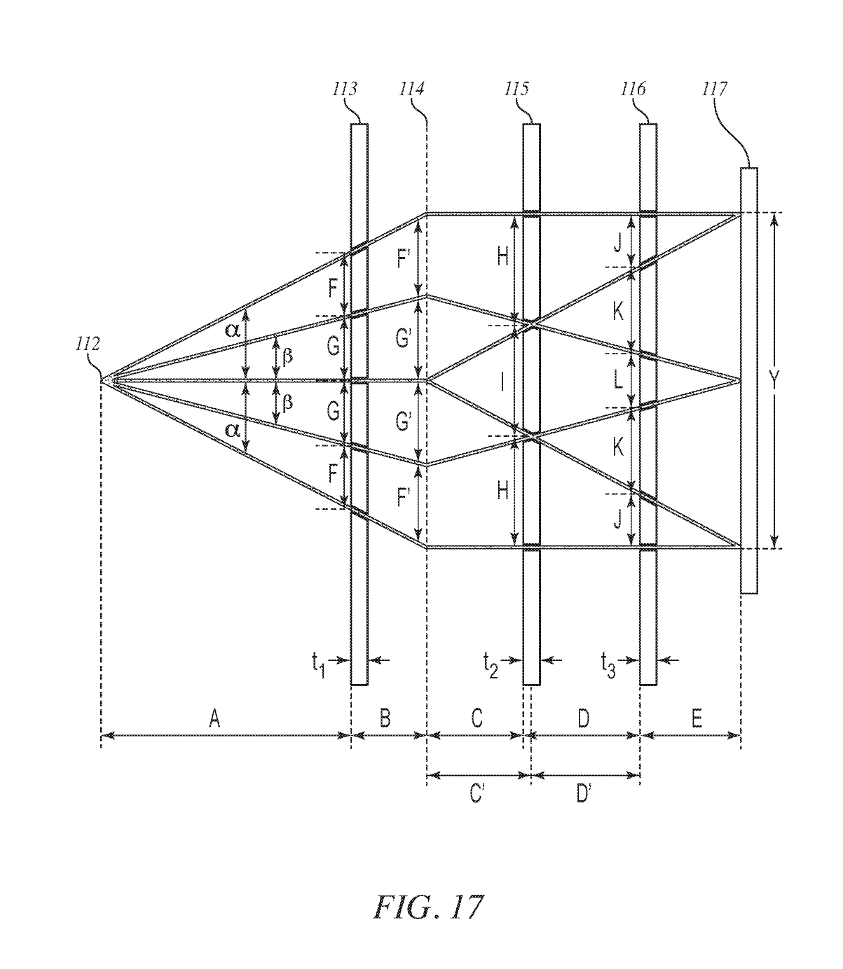

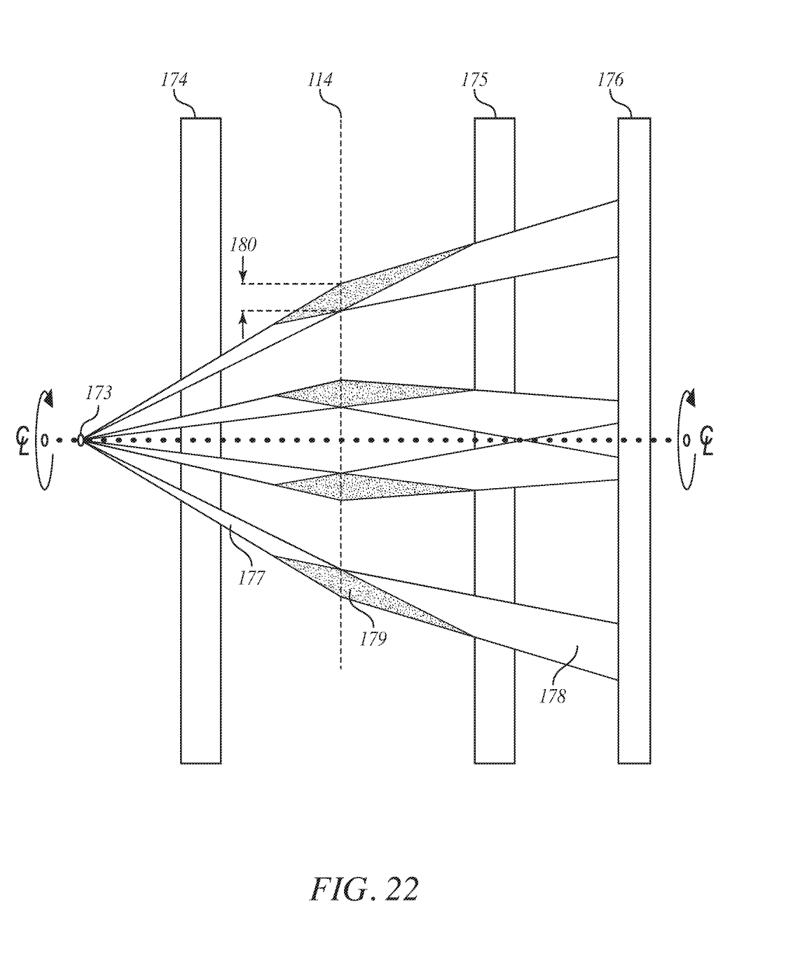

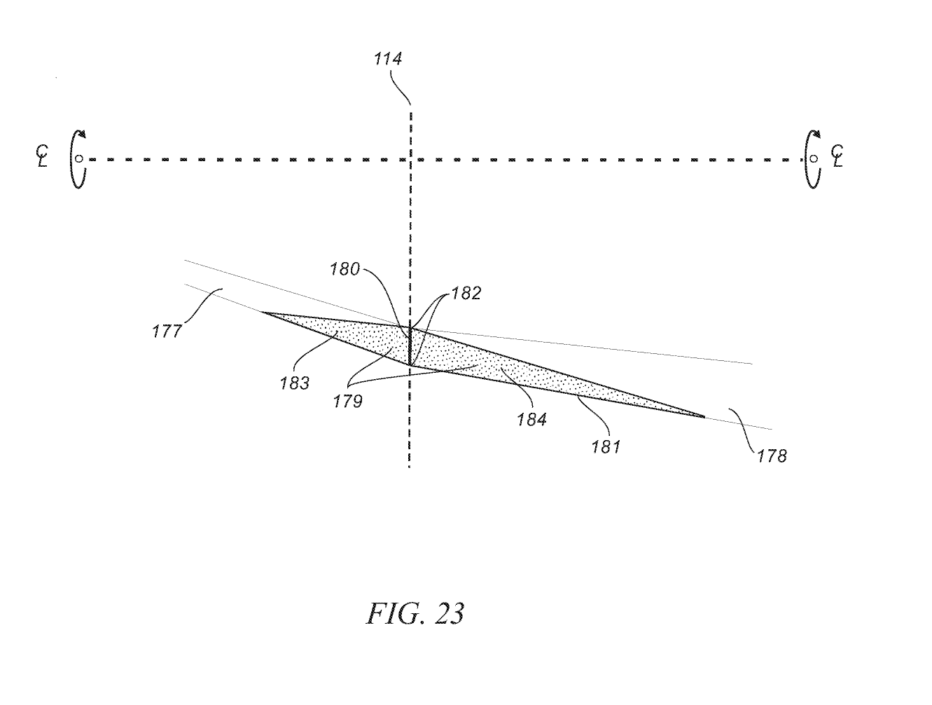

The diffraction plane 114 shown in FIG. 17 is a key element of the present invention shown in FIG. 4, since diffracted rays must originate on that plane so that the plurality of the diffracted beams can be summed by the detector to increase the diffracted intensity without degrading the spectral resolution. FIG. 22 further illustrates how the diffraction plane relates to the diffraction geometry for a particular system design. The X-ray source 173 for this discussion is a point source and coincides with the apex of each of the annular slits in the incident beam collimator 174, resulting in a divergent incident beam of fixed angular width 177. After interacting with the sample (not shown), diffracted rays are created and some of these are able to pass through the slits in the receiving collimator 175, which may be either a single thick collimator or a pair of receiving collimators as previously discussed. One example of the diffracted beams that can successfully pass through the receiving collimator(s) and ignoring any penumbra effect is shown as 178. The region 179 is the intersection of the allowed incident beam paths 177 and the allowed diffracted beam paths 178 and is frequently called the voxel (volume element). In the longitudinal cross section shown in FIG. 22, the voxel 179 has the shape of an irregular convex quadrilateral 181. By proper design of the slit opening and angle, the short axis 180 connecting the vertices 182 of each quadrilateral 181 will lie on a plane 114 perpendicular to the system symmetry axis as shown in detail in FIG. 23. Depending on the angle of the incident beam 177 and the acceptance angle of the diffracted beam 178, the individual contributions 183 and 184 to the diffraction voxel 179 may or may not be symmetric with respect to the diffraction plane 114. This requires that each of the annular rings in the diffracted beam collimator must be designed individually, especially with regard to the unequal angles for the top and bottom of the slit.

Another aspect of the present invention is an algorithm to compute the design parameters of an EDXRD system such as those previously described once the Bragg angle has been selected. One such example is illustrated in FIG. 17 that will demonstrate the key aspects of the algorithm for the design of an EDXRD system comprised of a point source of X-rays 112, an incident beam collimator 113, a first diffracted beam collimator 115, a second diffracted beam collimator 116 and a large area energy dispersive detector 117, To simplify the design for illustration purposes, the incident beam collimator contains only two annular slits 50 & 51 and a central pencil beam 49 as previously described in FIGS. 4 to 6. The first diffracted beam collimator 115 can be placed anywhere between the edge of the sample and the second diffracted beam collimator 116. However, the position C′ marking the center of the collimator 115 in FIG. 17 is preferred since this is the point where two of the diffracted beams cross. Selecting this point minimizes the number of slits that need to be machined. The position of the second diffracted beam collimator 116 can be anywhere between the first diffracted beam collimator 115 and the detector 117. The design shown in FIG. 17 has certain geometric constraints, such as 1) all incident/diffracted beam pairs have the same Bragg angle; 2) the uppermost and lowermost diffracted beams are parallel and separated by a distance Y that is no greater than the diameter of the active region of the detector 117; 3) the annular slits in the incident beam collimator 113 have the same apex, which coincides with the X-ray focal point 112; 4) the system is symmetric about the system axis; and 5) the pencil beam lies on the symmetry axis. Several of the variables are fixed including α, β, A, D, t₁, t₂, t₃, and Y, while the remaining variables can be calculated by the following formulae:

$$\begin{matrix}B = \frac{Y}{2 tan ( α )} - A & ( 2 ) \\ C = \frac{( A + B ) tan ( β )}{( tan ( α ) + tan ( β ) )} - \frac{{t}_{2}}{2} & ( 3 ) \\ {C}^{′} = \frac{( A + B ) tan ( β )}{( tan ( α ) + tan ( β ) )} & ( 4 ) \\ {D}^{′} = D - \frac{{t}_{2}}{2} & ( 5 ) \\ E = \frac{Y}{2 tan ( α )} - \frac{( A + B ) tan ( β )}{( tan ( α ) + tan ( β ) )} - {D}^{′} & ( 6 ) \\ F = A ( tan ( α ) - tan ( β ) ) & ( 7 ) \\ {F}^{′} = ( A + B ) ( tan ( α ) - tan ( β ) ) & ( 8 ) \\ G = A tan ( β ) & ( 9 ) \\ {G}^{′} = ( A + B ) tan ( β ) & ( 10 ) \\ H = \frac{Y}{2} - \frac{( A + B ) tan ( β )}{( tan ( α ) + tan ( β ) )} & ( 11 ) \\ I = \frac{2 ( A + B ) tan ( α ) tan ( β )}{( tan ( α ) + tan ( β ) )} & ( 12 ) \\ J = \frac{Y}{2} - \frac{( A + B ) tan ( α ) tan ( β )}{( tan ( α ) + tan ( β ) )} - {D}^{′} tan ( α ) & ( 13 ) \\ K = {D}^{′} ( tan ( α ) + tan ( β ) ) & ( 14 ) \\ L = \frac{2 ( A + B ) tan ( α ) tan ( β )}{( tan ( α ) + tan ( β ) )} - 2 {D}^{′} tan ( β ) & ( 15 )\end{matrix}$$

This method can be extended to include a greater or lesser number of incident annular incident beams, a diffracted beam collimator that combines the two diffracted beam collimators 115 and 116 as illustrated in FIG. 9, or a larger or smaller energy dispersive detector 117.

Another aspect of the present invention is an algorithm 118 to evaluate the performance of an EDXRD system design comprising a Monte Carlo method to estimate the overall system efficiency, the relative diffracted intensity and the spectral resolution based on the design parameters such as those described in eqs. 2-15 above. The algorithm also takes into account translation and tilt misalignments that may occur in assembling the apparatus and thus determines the sensitivity of a particular system design to typical manufacturing imperfections.

The EDXRD evaluating algorithm 118 comprises numerous functions to first establish the relative positions of the system components, apply misalignments as directed, generate randomly oriented incident X-ray beams and determine if the diffracted beams are able to pass through the various slits to reach the detector. Statistics are collected as each incident and diffracted beam interacts with each of the hardware components to determine the relative efficiency at every step. Those beams able to reach the detector are used to compute the resulting diffraction profile, its relative intensity and the system's resolving power.

The computer program used to implement the EDXRD evaluation algorithm 118 utilizes a Graphical User Interface (GUI) 120, 125 to control entry of the data input 121 & 122, computation of slit positions 123, applications of misalignments 124, modes of operation 126, generation of the randomly oriented incident X-ray beams, collection and analysis of statistics, graphical display 125 and archiving experimental results 134. The GUI 120, 125 allows the operator to enter the design parameters by hand or to load such data from a stored input file 128. The operator can also initialize all the variables 127 to start additional experiments 135. Once the EDXRD design data has been entered 122, the positions and angles of all slits in both the incident and diffracted beam collimators are computed 123. Misalignments of the various system components such as translation errors parallel to and perpendicular to the system symmetry axis and tilt errors can be included 124 using a conventional coordinate transformation process 129. Once the system geometry, including the misalignments have been computed, the operator can choose between a fast cycle mode 130, a mini-step mode 131, a leak check mode 132, and a full simulation mode 133. Two of the operational modes, the mini-step and the fast cycle mode are used to slow down the simulation and inspect one incident x-ray beam at a time, which acts as a tool to identify design flaws. In the mini-step mode 131, the simulation stops when a beam reaches each collimator slit so that the operator can visualize the beam paths. In the fast cycle mode 130, the simulation stops only when a beam reaches the detector or is blocked by one of the collimators. A third option, a full simulation mode 133, does not stop to allow the operator to see one beam at a time. Instead, the simulation runs to completion for all of the randomly generated incident beams and displays only the results of the statistical analysis, the computed diffraction profile and data such as diffracted pattern intensity and system resolving power. Finally, a fourth operational mode, leakage check 132, is available, where the sole purpose is to verify that no incident X-ray beam can directly reach the detector. If any leakage is found, an appropriate message is displayed and all data related to the leak is recorded and displayed 149. The GUI also offers other options such as “Save Results” 134, which allows the operator to select a data file for data storage, “Start Over” 135, which clears all the input data fields and restarts the program with a new data set, and “Exit Program” 136, which terminates the program without saving the output data.

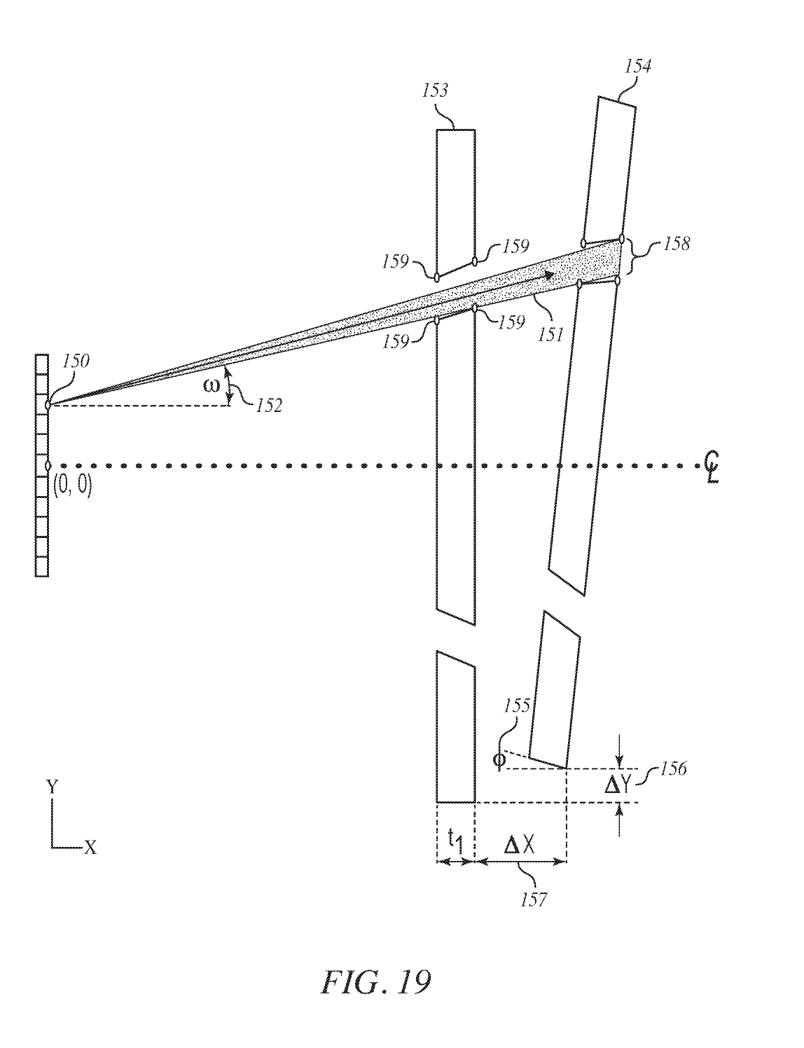

Within the simulation modes 130, 131, and 133, the program will generate an incident X-ray beam 151 from a random point on the X-ray source 150 and in a random direction 152 towards the incident beam collimator 153 as illustrated in FIG. 19. By using the angular acceptance range 158 of the slit opening, the program “C1 Passage” 137 can determine if the random ray can pass through the slits in the incident beam collimator 153. The angular acceptance range 158 is defined with respect to the particular point on the X-ray source 150 and the upper and lower openings on the slit for both the entrance and exit sides of the slit 159 after the tilt 155, vertical 156 and lateral 157 misalignments have been taken into account. If a beam successfully passes through the slit, then an appropriate counter is incremented to record which slit the beam has passed through and a record is also made of the angular direction ω of the beam 152, which is used later to determine the Bragg angle of a diffracted beam.

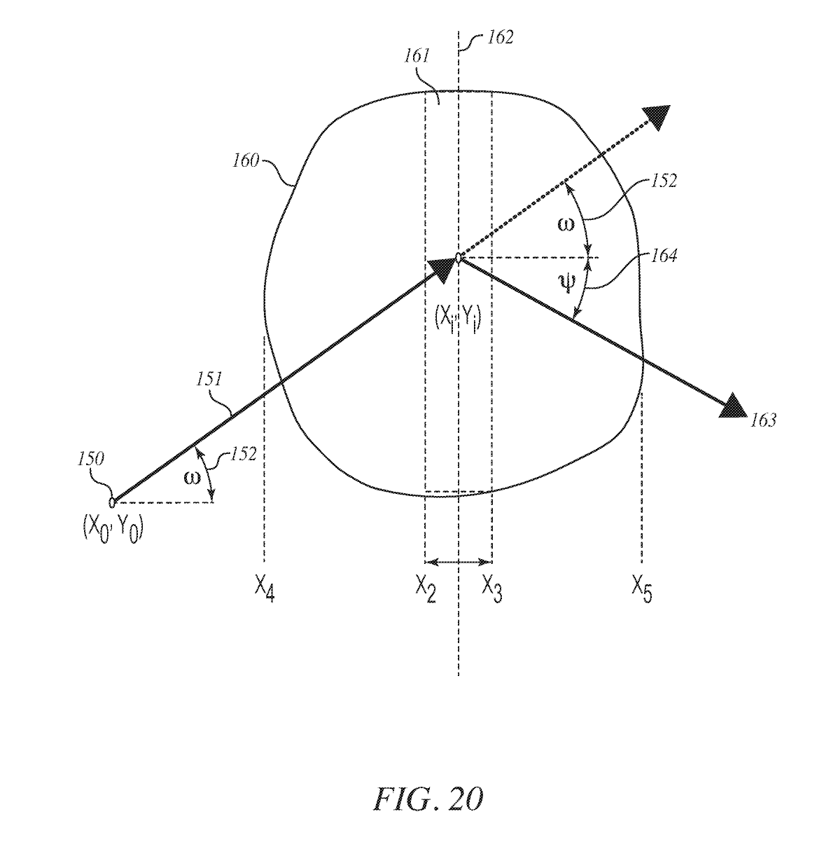

After the incident beam successfully passes through one of the slits in the incident beam collimator, the beam 151 continues on to interact with the test sample 160 as illustrated in FIG. 20. The sample is discretized by subdividing it into n slices, one of which 161 is shown in FIG. 20 as the i'th slice with thickness s given by s−(X₅−X₄)/(number of slices). From the intersecting point of the incident beam 151 with the diffraction plane 162, the coordinates (Xᵢ, Yᵢ) for the source of the potential diffracted beam 163 are determined by Xᵢ=(X₄−X₀)+s(i−0.5) and Yᵢ=(Xᵢ−X₀)tan(ω)+Y₀, where (X₀, Y₀) are the coordinates of the X-ray beam at its source 150 and ω is the angle 152 that was randomly chosen and is capable of passing thru the collimator slit 154. At the diffraction event origin (Xᵢ, Yᵢ), a diffracted beam 163 is generated in a random direction ψ(164). Accordingly, the Bragg angle 2Θᵢ, defined as the angle between the incident and diffracted beams is 2Θᵢ=ω+ω. This diffracted beam is then tested to see if it can pass through the various slits in the one or more receiving collimators.

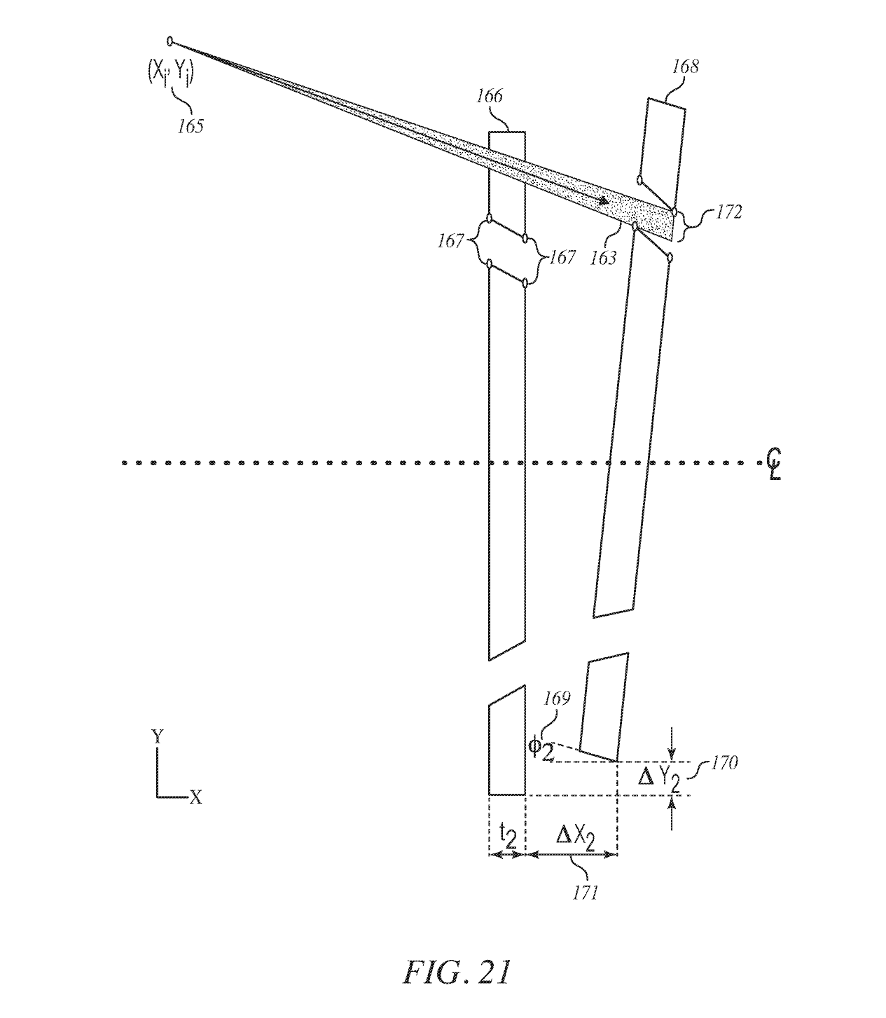

FIG. 21 illustrates the definition of the acceptance angle 172 for a diffracted beam 163 originating at the (Xᵢ, Yᵢ) point 165 in the discretized sample 160. As with the previous treatment of the incident beam collimator, the misalignment of the diffracted beam collimator is treated by tilting 169 and translating in the vertical 170 and lateral 171 directions. The four corners 167 that define the slit within the collimator 166 before misalignment shift to new values in the misaligned collimator 168. From the new (X,Y) coordinates of the misaligned slit openings, combined with the known values of the diffraction event origin (Xᵢ, Yᵢ), the acceptance angle can be computed and compared to the randomly oriented diffracted beam 163 to see if it passes through the receiving slit using the program segment “C2 Passage” 138. If the diffracted beam successfully passes through the slits in the misaligned first receiving collimator 168, an appropriate counter is incremented and both the incident beam angle ω and the diffracted beam angle are recorded.

Once a random diffracted beam has passed the first receiving collimator 168, an equivalent process is used to determine if the same beam 163 passes through the second diffracted beam collimator 46, 116 using the program segment “C3 Passage” 139. After passing through both diffracted beam collimators, a final test “Detector Passage” 140 is conducted to see if the beam intersects the detector. If so, all of the data related to the diffracted beam and the related incident beam are recorded 146 for later use in constructing the diffraction pattern 147.

After all of the randomly oriented beams from the X-ray source have been evaluated, the rays that were able to reach the detector and previously stored are used to construct the energy dispersive diffraction profile 147. Each ray reaching the detector will have a Bragg angle that is close to the design value, but will deviate to some degree depending on the size of the slit opening. Very narrow slits will result in a minimal dispersion of the Bragg angle, while wider slits will result in correspondingly larger dispersions. This dispersion has a direct relationship to the system's spectral resolving power, measured by the peak breadth at one half of the maximum intensity (FWHM) of a well resolved peak 148.

The EDXRD system evaluator also contains a number of housekeeping tools such as a timer 141 to record the elapsed time, a redraw function 142 to erase the non-numerical portion of the screen and redraw the ray tracing graphics, a screen dump feature 143, and a reset function 144, 145 to stop the simulation and reset.

The Monte Carlo method used in the EDXRD system evaluator is independent of the number of collimators, the number of slits within each collimator, the size of the X-ray source and detector, since all these data are entered at execution time. As a result, the program can be used to evaluate any of the systems of the present invention.

Various changes could be made in the above system and method without departing from the scope of the invention as defined in the claims below. It is intended that all matter contained in the above description, as shown in the accompanying drawings, shall be interpreted as illustrative and not as a limitation.

1. A system for identification of crystalline materials having a symmetry axis extending from the center of the X-ray source to the center of the detector face, comprising:
a. an X-ray source whose center is aligned with the system symmetry axis, the X-ray source emitting a polychromatic X-ray beam;
b. an incident beam collimator having a plurality of incident annular slits, each said incident annular slit having an apex, the incident beam collimator being aligned with and perpendicular to the system symmetry axis and each said incident annular slit having the X-ray source at its apex, wherein each said incident annular slit only permits passage of incident X-ray beams;
c. one or more diffracted beam collimators, each having a plurality of diffracted annular slits, the diffracted beam collimators being concentric with and perpendicular to the system symmetry axis, wherein the diffracted slits only permit passage of diffracted X-ray beams that originate from a diffraction plane perpendicular to the system symmetry axis, wherein the diffraction plane comprises vertices of a plurality of irregular convex quadrilateral cross-sections, the cross-sections being formed where the incident X-ray beam and a projection of an acceptance angle intersect, the acceptance angle being formed by the one or more diffracted beam collimators; and
d. an energy dispersive X-ray detector,
wherein the symmetry axis extends from the center of the X-ray source to the center of the detector face.

2. The system for identification of crystalline materials of claim 1, wherein the X-ray source is an air-cooled or oil-cooled tube operating at a power level of less than 200 Watts.

3. The system for identification of crystalline materials of claim 1, wherein the energy dispersive X-ray detector operates at or near −10° C. to 35° C.

4. The system for identification of crystalline materials of claim 1, wherein the energy dispersive X-ray detector is made from one or more compound semiconductors comprising CdTe, CdZnTe, HgI₂ and/or GaAs.

5. The system for identification of crystalline materials of claim 1, wherein the energy dispersive X-ray detector has an active detector area of at least 100 mm².

6. The system for identification of crystalline materials of claim 1, wherein the system further comprises one or more tangential collimators inserted before or after the first of the one or more diffracted beam collimators, wherein the tangential collimator contains a plurality of plates that are evenly distributed about and are joined at the system's symmetry axis, and wherein the tangential collimator reduces tangential divergence of the incident X-ray beams.

7. The system for identification of crystalline materials of claim 6, wherein the tangential collimator is combined in a monolithic structure with the one or more diffracted beam collimators.

8. The system for identification of crystalline materials of claim 6, wherein the diffracted beam collimators contain keyways and alignment holes to permit attachment of said collimators to alignment devices such as gimbals or cylindrical tubes containing matching keyways.

9. The system for identification of crystalline materials of claim 1, wherein the collimators are made from tungsten, tungsten carbide, or tungsten alloys.

10. The system for identification of crystalline materials of claim 1, wherein the collimators are made from chromium, manganese, iron, cobalt, nickel, copper, zinc, or alloys of chromium, manganese, iron, cobalt, nickel, copper, or zinc.

11. The system for identification of crystalline materials of claim 1, wherein the incident beam collimator contains a center circular hole aligned with the system symmetry axis.

12. The system for identification of crystalline materials of claim 1, wherein the incident beam collimator and the diffracted beam collimator(s) are manufactured by one or more processes comprising powder bed laser melting, direct metal laser melting, selective laser melting, and/or laser deposition and electron beam melting.

13. The system for identification of crystalline materials of claim 1, wherein
the incident beam collimator has one incident annular slit having an apex, the incident annular slit being aligned with and perpendicular to the system symmetry axis and the incident annular slit has the X-ray source at its apex; and wherein
the system comprises a diffracted beam collimator having a plurality of diffracted annular slits concentric with and perpendicular to the system symmetry axis in which said diffracted annular slits are parallel to each other.

14. The system for identification of crystalline materials of claim 13, wherein the X-ray source has a focal spot with dimensions greater than 20 mm².

15. The system for identification of crystalline materials of claim 14 wherein the incident beam collimator contains two or more annular slits that are parallel to each other and are concentric aligned with and perpendicular to the system symmetry axis and the incident annular slits do not have the X-ray source at their apexes of said annular slits are not coincident with the X ray source.

16. The system for identification of crystalline materials of claim 13 wherein the incident beam collimator and the diffracted beam collimators are manufactured by one or more processes comprising powder bed laser melting, direct metal laser melting, selective laser melting, and/or laser deposition and electron beam melting.

17. A system for identification of crystalline materials having a symmetry axis, comprising:
a. an X-ray source coincident with the system symmetry axis, the X-ray source emitting a polychromatic X-ray beam;
b. an incident beam collimator having a pinhole slit aligned with the system symmetry axis;
c. a diffracted beam collimator having a plurality of diffracted annular slits, the diffracted annular slits being concentric with and perpendicular to the system symmetry axis, and wherein said diffracted annular in which said slits are parallel to each other; and
d. an energy dispersive X-ray detector.

18. The system for identification of crystalline materials of claim 17, wherein the X-ray source has a focal spot with dimensions greater than 20 mm².

19. The system for identification of crystalline materials of claim 18 wherein the incident beam collimator comprises two or more pinhole slits that are parallel to each other and parallel to the system symmetry axis.

20. The system for identification of crystalline materials of claim 17, wherein the incident beam collimator and the diffracted beam collimators are manufactured by one or more processes comprising powder bed laser melting, direct metal laser melting, selective laser melting, and/or laser deposition and electron beam melting.

21. A method of identification of crystalline materials by X-ray diffraction, comprising:
a. irradiating a crystalline material with a broad spectrum X-ray beam, the X-ray beam being emitted by an X-ray source in a system having a symmetry axis;
b. collecting a plurality of incident X-ray beam by means of an incident beam collimator having a plurality of incident annular slits, each having incident annular slit having an apex, the incident beam collimator being concentric with and perpendicular to the system symmetry axis and each said incident annular slit having the X-ray source at its apex;
c. collecting a plurality of diffracted X-ray beams by means of one or more diffracted beam collimators having a plurality of diffracted annular slits, the diffracted beam collimators being concentric with and perpendicular to the system symmetry axis, wherein the diffracted annular slits only permit passage of the diffracted X-ray beams that originate from a diffraction plane perpendicular to the system symmetry axis, wherein the diffraction plane comprises vertices of a plurality of irregular convex quadrilateral cross-sections, the cross-sections being formed where the incident X-ray beams and a projection of an acceptance angle intersect, the acceptance angle being formed by the one or more diffracted beam collimators;
d. collecting and integrating the plurality of diffracted X-ray beams that pass through the diffracted beam collimators by means of a single energy dispersive X-ray detector; and
e. comparing the X-ray diffraction pattern collected by the detector and matching said diffraction pattern to a library of known materials.

22. The method of claim 21, wherein the crystalline material comprises a storage container enclosing pharmaceuticals.

23. The method of claim 21, wherein the crystalline material comprises a storage container enclosing counterfeit pharmaceuticals.

24. The method of claim 21, wherein the crystalline material comprises a storage container enclosing subpotency pharmaceuticals.

25. The method of claim 21, wherein the crystalline material comprises pharmaceuticals which have undergone a polymorphic transformation during storage or manufacture.

26. The method of claim 21, wherein the crystalline material comprises a blister pack containing pharmaceuticals.

27. A method of designing an energy dispersive X-ray diffraction system using an instrument having physical features comprising design parameters of a collimator, slit spacings, and an X-ray source intensity and design values comprising a diffraction angle, a test sample size, a maximum detector size, and a focal spot size of the X-ray source, the system having parameters comprising a diffracted peak intensity, a spectral resolution, and translational and rotational misalignments, the method comprising
a. identifying a first diffraction angle for energy dispersive X-ray diffraction analysis by converting via computation a known angular dispersive X-ray diffraction pattern into an energy dispersive X-ray diffraction profile using Bragg's law of diffraction and then correcting the energy dispersive X-ray diffraction profile to account for the X-ray source intensity profile and then further correcting the energy dispersive X-ray diffraction profile by convoluting it with an instrument profile, the instrument profile being either an experimental or calculated profile that accounts for the physical features of the instrument, providing a resulting diffraction profile, followed by an analysis of the resulting diffraction profile to determine the number of strong diffraction peaks between 10 keV and 60 keV;
b. repeating the steps of a. for additional diffraction angles to find which diffraction angle results in the greatest number of strong diffraction peaks in the energy range of 10 keV to 60 keV;
c. calculating the design parameters of the collimator and slit spacings subject to geometrical constraints and symmetry operators using the fixed design values comprising the optimal diffraction angle, the test sample size, the maximum detector size, and the focal spot size of the X-ray source focal spot; and
d. evaluating the system's performance by using a Monte Carlo scheme to compute the system efficiency, the diffracted peak intensity, the system's spectral resolution and taking into account possible translational and rotational misalignment of the system.
